SEQUENCE LISTING (11 sequences)

SEQ ID NO: 1 - protein, 8 aa, Artificial Sequence
  Synthetic sequence
SIINFEKL

SEQ ID NO: 2 - protein, 8 aa, Artificial Sequence
  Synthetic sequence
XIINFEKL

SEQ ID NO: 3 - protein, 8 aa, Artificial Sequence
  Synthetic sequence
SXINFEKL

SEQ ID NO: 4 - protein, 8 aa, Artificial Sequence
  Synthetic sequence
SIXNFEKL

SEQ ID NO: 5 - protein, 8 aa, Artificial Sequence
  Synthetic sequence
SIIXFEKL

SEQ ID NO: 6 - protein, 8 aa, Artificial Sequence
  Synthetic sequence
SIINXEKL

SEQ ID NO: 7 - protein, 8 aa, Artificial Sequence
  Synthetic sequence
SIINFXKL

SEQ ID NO: 8 - protein, 8 aa, Artificial Sequence
  Synthetic sequence
SIINFEKX

SEQ ID NO: 9 - protein, 9 aa, Artificial Sequence
  Synthetic sequence
QLSLLMWIT

SEQ ID NO: 10 - protein, 9 aa, Artificial Sequence
  Synthetic sequence
SLLMWITQC

SEQ ID NO: 11 - protein, 11 aa, Artificial Sequence
  Synthetic sequence
SLLMWITQCFL

(19) United States
(12) Patent Application Publication
(10) Pub. No.: US 2006/0122107 A1
(43) Pub. Date: Jun. 8, 2006

(54) Method of modulating cellular activity and molecules for use therein

(75) Inventors: Marie-Isabel Aguilar, North Warrandyte (AU); Patrick Perlmutter, Elsternwick (AU); Anthony Purcell, Macleod (AU); Andrew Webb, Mooroolbark (AU); Jamie Rossjohn, Carnegie (AU)

Correspondence Address: SEED INTELLECTUAL PROPERTY LAW GROUP PLLC, 701 FIFTH AVE, SUITE 6300, SEATTLE, WA, 98104-7092, US

(73) Assignees: Monash University, Clayton (AU); The University of Melbourne, Parkville (AU)

(21) Appl. No.: 10/526,829

(22) PCT Filed: Sep. 4, 2003
(86) PCT No.: PCT/AU03/01150

§ 371 Date: Sep. 26, 2005

(30) Foreign Application Priority Data
Sep. 4, 2002 (AU) 2002951212

(51) Int. Cl. A61K 38/16; A61K 38/17

(52) U.S. Cl. 514/12

(57) ABSTRACT

The present invention relates generally to a method of modulating T cell functional activity by utilising β-amino acid substituted peptides and to agents useful for the same. More particularly, the present invention relates to a method of modulating class I restricted T cell activity by utilising β-amino acid substituted peptides and to agents useful for the same. The method of the present invention is useful, inter alia, in the treatment and/or prophylaxis of conditions characterised by suboptimal T cell stimulation such as that which occurs in some viral infections and in anti-tumour immunity, as well as aberrant, unwanted or otherwise inappropriate T cell functioning such as, but not limited to, graft rejection or autoimmune conditions, the present invention is further directed to methods of identifying, designing and/or modifying agents capable of modulating T cell functional activity.

### FIELD OF THE INVENTION

The present invention relates generally to a method of modulating T cell functional activity and to agents useful for same. More particularly, the present invention relates to a method of modulating class I restricted T cell activity and to agents useful for same. The method of the present invention is useful, inter alia, in the treatment and/or prophylaxis of conditions characterised by suboptimal T cell stimulation such as that which occurs in some viral infections and in anti-tumour immunity, as well as aberrant, unwanted or otherwise inappropriate T cell functioning such as, but not limited to, graft rejection or autoimmune conditions. The present invention is further directed to methods of identifying, designing and/or modifying agents capable of modulating T cell functional activity.

### BACKGROUND OF THE INVENTION

The Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia.

Class I major histocompatability complex (MHC) molecules play a crucial role in immune surveillance by selectively binding to intracellular peptide antigens and presenting them at the cell surface to cytotoxic T lymphocytes.

Peptide antigens that bind to MHC molecules, whether they be MHC class I or class II molecules, are first liberated from intact proteins. The compartment within a cell where the antigen is processed determines whether it will meet MHC class I or class II molecules. MHC class I molecules usually bind peptides from proteins which are normally resident in, or have been delivered to, the cytoplasm, whereas MHC class II molecules bind peptides mostly derived from proteins internalised into acidic vesicles from the extracellular environment. Peptides are generated continuously in a cell by proteolysis, principally in the cytoplasm of the cell by a complex known as the proteasome. This results in a wide range of suitable peptide substrates for MHC class I molecules. Once peptides are generated, they must traverse the endoplasmic reticulum (ER) membrane in order to associate with the MHC molecule.

MHC class I molecules are comprised of a light chain, known as β-2 microglobulin, complexed with a single heavy chain. The structure of the class I molecule is suited to capture shorter peptides for presentation to cytotoxic T lymphocytes. The MHC class I molecule is comprised of three main domains, being the α1, α2 and α3 domains. The relatively conserved structured of the α3 domain interacts with CD8 molecules present on the surface of cytotoxic T lymphocytes. The outermost domains (α1 and α2) make up two long α helices separated by a cleft with a floor composed of a β pleated sheet. This cleft forms the antigen binding pocket of the MHC class I molecule, and is the major site for structural polymorphisms (Yuan-Hua Ding, Brian M. Baker, David N. Garboczi. Four A6-TCR/Peptide/HLA-A2 Immunity, Vol. 11, 45-56, July, 1999). Polymorphisms of this cleft result in changes of the electrostatic charge and shape of the floor and walls of the cleft, which in turn result in changes to the peptide binding properties of individual MHC alleles. This allows the MHC class 1 molecule to bind a unique repertoire of endogenous peptides.

Cytotoxic T lymphocytes recognise fragments of protein antigens presented by the MHC molecules of target cells through their T cell receptor. The T cell receptor is a heterodimer made up of an α and β chain. Both chains comprise variable and constant domains. The variable domain contains loops known as complimentary determining regions which dominate the surface that makes contact with the peptide-MHC complex. Cytotoxic T lymphocytes kill target cells via at least two known distinct pathways. The secretory pathway involving exocytosis of cytolytic granules and the Fas pathway, in which the Fas ligand on the CTL surface interacts with the Fas receptor on the target cell. Both these mechanisms appear to work independently to induce apoptosis culminating in destruction of the target cell.

Signalling through membrane bound TCR complex is necessary for the development of the T cell repertoire and the initiation of a cellular immune response. Currently, little is known about the mechanisms of signal transduction once the T cell receptor is bound by its ligand. However, it is believed that this mechanism involves activation of tyrosine kinases and other potential effector molecules in favor of signal transduction. Nevertheless, there remain a number of competing models which purport to define the functioning of the signal mechanism. In this regard, there is currently strong opinion that the properties of the T cell receptor-peptide MHC complex interaction determine the outcome of binding.

Cytotoxic T lymphocyte activation defines a highly sophisticated and crucial component of the immune system. Normally, an individual's cytotoxic lymphocytes are only activated by non-self peptides (such as peptides of viral origin), thereby triggering an immune response. The identification and appropriate delivery of such peptides underpins vaccine design for improved anti-viral vaccines and anti-tumour vaccines. The ability to enhance the stability of peptide-based vaccine components is highly desirable due to the rapid in vivo degradation of peptides and protein based constituents of such vaccines.

In certain aberrant situations the normal functioning of the immune system is compromised due to an individual's cytotoxic T lymphocyte becoming activated by self antigens. This leads to the development of a pathological state termed autoimmunity. Clearly, there is a need to develop means of modulating T cell functioning in order to obviate the sometimes very severe consequences of aberrant T cell functioning, such as the development of autoimmune conditions.

β-amino acids been recently recognised as a potential new peptidomimetic approach to the design of novel bioactive peptides. β-amino acids are similar to α-amino acids in that they contain an amino terminus and a carboxyl terminus. However, two carbon atoms separate these functional terminii. β-amino acids have been demonstrated to achieve successful incorporation into amino acid chains such as to create peptidomimetics which exhibit biological activity. To date, there have been a number of studies directed to the use of β-amino acids in the context of peptidomimetics. However, the very limited studies which have been conducted in the context of MHC presentation have demonstrated only that some β-amino acid peptide analogues can successfully bind to the MHC cleft. Further, these studies have suggested that such modulation of peptide structure does not impact on T cell functioning.

One of the confounding issues associated with the use of peptides as vaccines is there rapid degradation post-vaccination. This arises from metabolism in the liver and other organs and by proteolysis. In addition, proteolysis of peptide vaccines can lead to the generation of cryptic epitopes, i.e. fragments of the intended immunogen that elicit futile or worse still unwanted immune responses. In work leading up to the present invention, it has been surprisingly determined that the substitution of β-amino acids for α-amino acids in the context of MHC class I peptides can, in fact, induce highly significant modulation of T cell functioning. In particular, both agonism and antagonism of the cytotoxic T cell response can be achieved. Still further, there has occurred the completely unexpected determination that peptides which either agonise or antagonise the cytotoxic T cell response in isolation may, when coadministered with unmodified peptide, result in overall upregulation of the converse response (that is antagonism or agonism, respectively) in respect of the unmodified peptide. The determination of these functional and structural relationships now facilitates the development of molecules and methods for the modulation of T cell responses and, more particularly, the treatment of conditions characterised by aberrant, unwanted or otherwise inappropriate T cell functioning.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” shall be taken to indicate that a particular integer or group of integers has originated from the species specified, but has not necessarily been obtained directly from the specified source.

The subject specification contains amino acid sequence information prepared using the programme PatentIn Version 3.1, presented herein after the bibliography. Each amino acid sequence identified in the sequence listing by the numeric indicator <201> followed by the sequence identifier (eg. <201>1, <201>2, etc). The length, type of sequence (protein, etc) and source organism for each sequence is indicated by information provided in the numeric indicator fields <211>, <212> and <213>, respectively. Amino acid sequences referred to in the specification are identified by the indicator SEQ ID NO: followed by the sequence identifier (eg. SEQ ID NO:1, SEQ ID NO:2, etc.). The sequence identifier referred to in the specification correlates to the information provided in numeric indicator field <400> in the sequence listing, which is followed by the sequence identifier (eg. <400>1, <400?2, etc). That is SEQ ID NO:1 as detailed in the specification correlates to the sequence indicated as <400>1 in the sequence listing.

One aspect of the present invention is directed to a method of modulating a peptide specific T cell response, said method comprising contacting said T cell with an MHC-peptide complex, which peptide comprises at least one β-amino acid substitution, wherein said β-amino acid substitution induces either agonism or antagonism of said T cell response relative to the T cell response inducible by a non-substituted form of said peptide.

Another aspect of the present invention provides a method of modulating a peptide specific CD8⁺ T cell response, said method comprising contacting said CD8⁺ T cell with an MHC I-peptide complex, which peptide comprises at least one β-amino acid substitution, wherein said β-amino acid substitution induces either agonism or antagonism of said CD8⁺ T cell response relative to the CD8⁺ T cell response inducible by a non-substituted form of said peptide.

Yet another aspect of the present invention provides a method of modulating peptide specific CD8⁺ T cell activation, said method comprising contacting said CD8⁺ T cell with an MHC I-peptide complex, which peptide comprises at least one β-amino acid substitution, wherein said β-amino acid substitution induces either agonism or antagonism of said CD8⁺ T cell activation relative to the CD8⁺ T cell activation inducible by a non-substituted form of said peptide.

Still another aspect of the present invention provides a method of modulating a peptide specific T cell response in a subject, said method comprising administering to said subject an effective amount of a peptide, which peptide comprises at least one β-amino acid substitution, for a time and under conditions sufficient to present said peptide to said T cell in the context of an MHC-peptide complex, wherein said β-amino acid substitution induces either agonism or antagonism of said T cell response relative to the T cell response inducible by a non-substituted form of said peptide.

Still yet another aspect of the present invention provides a method of modulating a peptide specific cytotoxic T cell response in a subject, said method comprising administering to said subject an effective amount of a peptide, which peptide comprises at least one β-amino acid substitution, for a time and under conditions sufficient to present said peptide to said cytotoxic T cell in the context of an MHC I-peptide complex, wherein said β-amino acid substitution induces either agonism or antagonism of said cytotoxic T cell response relative to the cytotoxic T cell response inducible by a non-substituted form of said peptide.

The present invention also contemplates a method for preventing unwanted immune responses towards cryptic epitopes generated by proteolysis of vaccine components, by substitution of at least one β-amino acid into said precursor peptide to prevent such proteolysis and prevent generation of said cryptic epitopes and their subsequent presentation to said T cells in the context of an MHC-peptide complex.

Yet still another aspect of the present invention provides a means of agonising a peptide specific T cell response in a subject, said method comprising co-administering to said subject an effective amount of an antagonistic peptide, which peptide comprises at least one β-amino acid substitution, together with the non-substituted form of said peptide for a time and under conditions sufficient to present said peptides to said T cells in the context of an MHC-peptide complex.

In yet another aspect there is provided the method of antagonising a peptide specific T cell response in a subject, said method comprising co-administering to said subject an effective amount of an agonistic peptide, which peptide comprises at least one β-amino acid substitution, together with the non-substituted form of said peptide for a time and under conditions sufficient to present said peptides to said T cells in the context of an MHC-peptide complex.

The present invention also contemplates a method for the treatment and/or prophylaxis of a condition characterised by an aberrant, unwanted or otherwise inappropriate peptide specific T cell response in a subject, said method comprising administering to said subject an effective amount of a peptide, which peptide comprises at least one β-amino acid substitution, for a time and under conditions sufficient to present said peptide to said T cell in the context of an MHC-peptide complex, wherein said β-amino acid substitution induces either agonism or antagonism of said T cell response relative to the T cell response inducible by a non-substituted form of said peptide.

Another aspect of the present invention provides a method for the treatment and/or prophylaxis of a condition characterised by the occurrence of an unwanted peptide specific T cell response in a subject, said method comprising co-administering to said subject an effective amount of an agonistic peptide, which peptide comprises at least one β-amino acid substitution together with a non-substituted form of said peptide for a time and under conditions sufficient to present said peptides to said T cells in the context of an MHC peptide complex.

In still another aspect there is provided a method for the treatment and/or prophylaxis of a condition characterised by an inadequate peptide specific T cell response in a subject, said method comprising co-administering to said subject an effective amount of an antagonist peptide, which peptide comprises at least one β-amino acid substitution, together with a non-substituted form of said peptide for a time and under conditions sufficient to present said peptides to said T cells in the context of an MHC peptide complex.

In still another aspect, the present invention contemplates the use of a β-amino acid substituted peptide as hereinbefore defined in the manufacture of a medicament for the treatment of a condition in a mammal, which condition is characterised by an aberrant, unwanted or otherwise in appropriate T cell response wherein said β-amino acid substitution induces either agonism or antagonism of said T cell response relative to the T cell response inducible by a non-substituted from of said peptide.

In yet another further aspect, the present invention contemplates a pharmaceutical composition comprising the modulatory agent as hereinbefore defined and one or more pharmaceutically acceptable carriers and/or diluents. Said agents are referred to as the active ingredients

In yet another aspect, there has been developed a method of designing and screening for β-amino acid substituted peptide analogues, which method provides a means of rationally substituting α-amino acids for β-amino acids in a positional scanning approach and the identification of peptides exhibiting desired functional activity and improved bioavailability. The subject method is based on generating a population of peptide analogues by the sequential substitution of one or more of the α-amino acids comprising the peptide of interest with the corresponding β-amino acid and functionally analysing the analogues derived therefrom. Accordingly, it should be understood that the present invention extends to this screening method and the peptides derived therefrom.

Amino acid structure and single and three letter abbreviations used throughout the specification are defined in Table 1.

TABLE 1

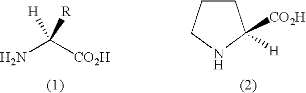

| | Three-letter | One-letter | |
|---|---|---|---|
| Amino Acid | Abbreviation | symbol | Structure of side chain (R) |
| Alanine | Ala | A | —CH₃ |
| Arginine | Arg | R | —(CH₂)₃NHC(═N)NH₂ |
| Asparagine | Asn | N | —CH₂CONH₂ |
| Aspartic acid | Asp | D | —CH₂CO₂H |
| Cystine | Cys | C | —CH₂SH |
| Glutamine | Gln | Q | —(CH₂)₂CONH₂ |
| Glutamic acid | Glu | E | —(CH₂)₂CO₂H |
| Glycine | Gly | G | —H |
| Histidine | His | H | —CH₂(4-imidazolyl) |
| Isoleucine | Ile | I | —CH(CH₃)CH₂CH₃ |
| Leucine | Leu | L | —CH₂CH(CH₃)₂ |
| Lysine | Lys | K | —(CH₂)₄NH₂ |
| Methionine | Met | M | —(CH₂)₂SCH₃ |
| Phenylalanine | Phe | F | —CH₂Ph |
| Proline | Pro | P | see formula (2) above for |
| | | | structure of amino acid |
| Serine | Ser | S | —CH₂OH |
| Threonine | Thr | T | —CH(CH₃)OH |
| Tryptophan | Trp | W | —CH₂(3-indolyl) |
| Tyrosine | Tyr | Y | —CH₂(4-hydroxyphenyl) |
| Valine | Val | V | —CH(CH₃)₂ |

### BRIEF DESCRIPTION OF THE DRAWINGS

FIG. 1 is a graphical representation of the staining of RMA-S cells with the H2-K^{b} specific monoclonal antibody Y-3. Data is shown as cell counts against fluorescence intensity.

FIG. 2 is a graphical representation of the staining of CTL clones with the monoclonal anti-CD8 antibody that is specific for CD8 molecules. Data is shown as cell counts against fluorescence intensity.

FIG. 3 is a graphical representation of the staining of I-3 cells with the H2-K^{b} specific monoclonal antibody Y-3. Data is shown as cell counts against fluorescence intensity.

FIG. 4 is a graphical representation of cell proliferation measured by ³H-Thymidine incorporation with increasing amounts of exogenous IL-2. Data shows the IL-2 dependence of CTLL-2 cells.

FIG. 5 is a graphical representation of K^{b} stabilisation on RMA-S by β_{C3}-amino acid substituted and parental peptides. Substitution at the N-terminal region of the peptide (a) disrupts K^{b} stabilisation, whereas substitution in the remaining middle and C-terminal end (b) results in equivalent or increased stabilisation. Serial dilutions of the indicated peptides were pulsed onto cold-induced RMA-S cells (26° C.) which were then pulsed at 37° C. Stabilisation of K^{b} at the surface was detected using the monoclonal antibody Y-3 and analysed by flow cytometry. Data is shown as percentage of maximal SIINFEKL response against peptide concentration (log scale).

FIG. 6 is a graphical representation of K^{b} stabilisation on RMA-S by β_{C3}-amino acid substituted and parental peptide using the antibody 25-D1.16. Substitution of β-amino acids resulted in a decreased affinity for the K^{b}/SIINFEKL specific antibody 25-D1.16. Data is shown as percentage of maximal wild type response against peptide concentration (log scale).

FIG. 7 is a graphical representation of the titration of Parental SIINFEKL against each CTL clone. As expected HSV2.3 control did not recognise the SIINFEKL peptide. Data is shown as radioactive counts against peptide concentration.

FIG. 8 is a graphical representation of the recognition of the parental SIINFEKL and β_{C3}-amino acid analogues by CTL clones. Substitution at P2 resulted in a significant loss of recognition with all clones except for GA4.2. At P6 the β_{C3}-Glu containing the benzyl side-chain protecting group completely abolished CTL recognition. Peptides were pulsed onto I-3 cells, then co-cultured with CTLs. Supernatant was then removed and added to an IL-2 bioassay. Data is shown as percentage of SIINFEKL response (in the case of the control clone, HSV2.3, the data is shown as percentage of SSIEFARL response).

FIG. 9 is a graphical representation of the recognition of the parental SIINFEKL and β_{C3}-amino acid analogues by CTL clone GA 4.2

FIG. 10 is a graphical representation of an antagonist assay using the CTL clones (a) GA4.2 and (b) B3.1. β-analogues are co-administered with a suboptimal concentration of the parental SIINFEKL peptide (dashed line). Supernatant of pulsed I-3 cells co-cultured with CTLs is then used in an IL-2 bioassay. Substitution at position P1 resulted in strong antagonism of B3.1 and weak antagonism of GA4.2. Substitution at P2 resulted a super agonist effect occurring in both clones. Both analogues mediated their response at extremely low concentrations indicating that only very low levels of analogue peptide are needed to alter signalling through the TCR.

FIG. 11 is a graphical representation of a serum stability assay showing the percentage of recovered peptide after 2 hrs in mouse serum. Peptides modified at P5 and P8 produced the most stable molecules.

FIG. 12 is a graphical representation of parental SIINFEKL degradation after 2 hrs as monitored by RP-HPLC at a wavelength of 214 nm. The Chromatogram shows the fragmentation of the parental peptide. The MS results reveal the cleavage sites for the enzymatic degradation. Cleavage occurs at bonds marked *:
S*I*I*NF*E*KL

FIG. 13 is a graphical representation of the degradation of the peptide containing a β-Phe at P5 at time points 0, 2 and 12 hrs. At t=0 the full peptide can be seen. At t=2 hrs the peptide full peptide is reduced and other fragments can be seen. At t=12 hrs the full peptide has disappeared leaving only fragments. MS of the fragments reveals that cleavage did not occur after the P5 amino acid as it did for the parental peptide. * Indicates blocked cleavage site:
S*I*I*NF*E*KL

FIG. 14 is a graphical representation of the degradation of the peptide containing a β-Ile at P3 at time points 0 and 2 hrs. At t=0 the full peptide can be seen. At t=2 hrs most of the full peptide has disappeared, with three main fragments. MS of the fragments revealed that there was no cleavage at the P2-P3 bond, which was a site of cleavage for the parental peptide. * Indicates blocked cleavage site:
S*I*I*NF*E*KL

FIG. 15 is a schematic representation of the presentation of natural and cryptic NY-ESO determinants following vaccination with NY-ESO 157-165 and 157-167 (taken from ¹).

FIG. 16 is a schematic representation of: A) HLA A*0201/ESO₁₅₇₋₁₆₅ complex crystallizes in cubic form. B) 2.1 Å electron density omit map of ESO₁₅₇₋₁₆₅ peptide. C) Cut-away view of ESO₁₅₇₋₁₆₅ bound to the HLA-A2 antigen binding cleft, highlighting the exposed Met-4, Trp-5, Thr-7, Gln-8.

FIG. 17 is a representation of a chromium release assay performed pursuant to in vivo priming.

FIG. 18 is a graphical representation of the intracellular cytokine staining of CTL-lines.

FIG. 19 is a schematic representation of a SIINFEKL peptide conformation.

FIG. 20 is a schematic representation of the SIIN-βF-EKL peptide conformation.

FIG. 21 is a schematic representation of the SIINFEK-βL peptide conformation.

### DETAILED DESCRIPTION OF THE INVENTION

The present invention is predicated, in part, on the surprising determination that β-amino acid substitution of MHC presented peptides can induce modulation of T cell functioning relative to the T cell functioning which is induced in respect of unmodified peptide. Further, it has still more surprisingly been determined that in addition to directly modulating the functioning of the T cell with which it interacts, in the context of MHC presentation, a β-amino acid substituted MHC presented peptide can also act to modulate the functioning of T cell populations which have interacted with unmodified peptides. Accordingly, these determinations now facilitate the development of molecules and methods for the modulation of T cell responses and, more particularly, for the treatment of conditions characterised by aberrant, unwanted or otherwise inappropriate T cell responses.

Accordingly, one aspect of the present invention is directed to a method of modulating a peptide specific T cell response, said method comprising contacting said T cell with an MHC-peptide complex, which peptide comprises at least one β-amino acid substitution, wherein said β-amino acid substitution induces either agonism or antagonism of said T cell response relative to the T cell response inducible by a non-substituted form of said peptide.

As detailed above, the present invention is predicated on the development of a method of modulating the qualitative and/or quantitative nature of a T cell response to a peptide, based on substituting one or more of the α-amino acids comprising the peptide with the corresponding β-amino acid. In this regard, it should be understood that although the present invention has been exemplified with respect to the modulation of the cytotoxic T cell response to an MHC I-presented peptide, this is not intended as a limitation on the scope of the invention. The teachings and principles detailed herein are applicable to any type of peptide restricted T cell response such as the MHC II restricted CD4 (T helper) cell response or the MHC I restricted CD8 (T cytotoxic) cell response.

Accordingly, reference to a “T cell” should be understood as a reference to any cell comprising a T cell receptor. In this regard, the T cell receptor may comprise any one or more of the α, β, γ or δ chains. The present invention is not intended to be limited to any particular function or sub-class of T cells, although in a preferred embodiment the subject T cell is a cytotoxic T cell.

Reference to “MHC” should be understood as a reference to any MHC molecule, such as MHC class I, II and/or IB molecules, and to all forms of these molecules. Without limiting the present invention to any one theory or mode of action, the major histocompatibility complex is a cluster of genes (on human chromosome 6 or mouse chromosome 17, for example) which encodes the MHC molecule. The MHC class I molecules are proteins which present peptides generated in the cytosol to CD8⁺ T cells (cytotoxic T cells) while the MHC class II molecules are proteins which present a peptide degraded in cellular vesicles to CD4⁺ T cells (helper T cells). The MHC is one of the most polymorphic gene clusters in the human genome, expressing large numbers of alleles of several different loci. The MHC comprises MHC class I, MHC class II and MHC class IB molecules. Unlike MHC class I and class II, the MHC class IB molecules are thought to present a restricted set of antigens and do not exhibit the same degree of polymorphism that the class I and class II genes exhibit.

In addition to encompassing the various polymorphic forms of MHC molecules which result from the expression of the unique MHC gene alleles, reference to “MHC” should also be understood to encompass all other mutant and polymorphic forms of MHC, such as any isoforms which arise from alternative splicing of MHC mRNA. Further, it should be understood that there is encompassed herein functional derivatives, homologues, analogues, equivalents and mimetics of the MHC molecules. For example, to the extent that a given vaccine strategy may involve peptide presentation by an exogenously introduced cell population (such as a cell population which has been loaded with antigen in vitro and reintroduced to a patient), but which population may also have undergone molecular or chemical modification at the level of its MHC molecules in order to facilitate improved presentation, such MHC molecules are encompassed herein. In another example, the subject MHC molecule may be an MHC homologue in that it is derived from an individual or even species distinct from the individual being treated. In this regard, although T cell activation is generally regarded as strictly restricted in the context of both peptide-MHC recognition by the TCR and in the context of host MHC presentation, there have been identified anomalous situations such as the form of presentation which occurs in relation to allogeneic or xenogeneic transplantation. In these situations, it is believed that the tissue rejection which occurs in the absence of immunosuppression is at least partly attributable to the presentation of donor peptides to recipient T cells in the context of donor MHC I and II. Although the basis for this phenomenon has not been fully elucidated, a certain degree of “leakage” does appear to occur in the context of host MHC restriction, and is therefore highly relevant in terms of transplantation technology. Further, the present invention should also be understood to encompass any other molecule which exhibits at least one of the functional characteristics of an MHC molecule. Such molecules include, for example, endogenously expressed molecules which exhibit MHC functional activity or molecules which have been introduced into the body (for example via a donor population of cells) and which mimic at least one of the MHC functions—such as the capacity to present a peptide to T cells.

Preferably, the subject T cell is a CD8⁺ T cell and said MHC molecule is an MHC I molecule.

The present invention therefore more particularly provides a method of modulating a peptide specific CD8⁺ T cell response, said method comprising contacting said CD8⁺ T cell with an MHC I-peptide complex, which peptide comprises at least one β-amino acid substitution, wherein said β-amino acid substitution induces either agonism or antagonism of said CD8⁺ T cell response relative to the CD8⁺ T cell response inducible by a non-substituted form of said peptide.

Reference to “peptide” should be understood as a reference to any molecule which comprises any number of contiguous or non-contiguous amino acids and which can be presented by an MHC molecule, preferably an MHC I molecule. By “presented” is meant that the peptide can interact with an MHC molecule and, more particularly, the MHC cleft.

An MHC molecule, the cleft of which is occupied by a peptide is herein referred to as a “MHC-peptide complex”. In this regard, and as hereinafter described, the MHC-peptide interaction may occur by any suitable means including as a result of intracellular processing and association with the MHC molecule or via extracellular delivery of the peptide to cell surface expressed MHC molecules. Preferably, the peptide comprises a contiguous sequence of 2-50 amino acids, more preferably 2-40 amino acids, still more preferably 2-30 amino acids, yet more preferably 2-20 amino acids and most preferably 2-15 amino acids.

In this regard, reference to a “peptide specific” T cell response should be understood as a T cell response which is induced as a consequence of T cell receptor interaction with a peptide-MHC complex. This should be contrasted, for example, with the extensive polyclonal T cell stimulation which can occur as a result of the actions of certain mitogens or superantigens, which achieve T cell stimulation by effectively circumventing the requirement for T cell activation to occur in an MHC restricted manner. However, it should be understood that the “peptide specific” T cell response of the present invention is any response which results from MHC presentation of a peptide. Due to the immunological specificity which is provided by the T cell receptor repertoire, any given peptide is likely to be specifically recognised (in the context of appropriate MHC presentation) by a very small number of T cell clones. However, due to the occurrence of certain degrees of crossreactivity, it is possible that additional T cell clones could also undergo stimulation in response to the presentation of such a peptide. It should be understood that any such T cell response (ie. whether it be a strong, weak, highly specific or cross reactive response) is a “peptide specific” T cell response in the context of the present invention due to it having resulted from the presentation of a peptide-MHC complex to a T cell receptor. In fact, in certain embodiments of the present invention, it may well be an objective to modify a peptide in accordance with the present teaching in order to strengthen the weak response of a given T cell clone, which results from crossreactivity, thereby improving a subject's overall immune response due to effectively increasing the number of T cell clones which can be expanded in response to a peptide of interest.

Reference to T cell “response” should be understood as a reference to any one or more of the functions which a T cell is capable of performing. For example, the subject response may be one or more of proliferation, differentiation (eg. induction of memory or effector T cells from virgin T cells) or other form of cellular activity such as the production of cytokines, upregulation of cell surface molecules, release of cytolytic granules (such as can occur with fully activated cytotoxic T cells) or intracellular signalling events. The present invention is directed to “modulating” this response, meaning that the response to the β-amino acid substituted peptide is either fully or partially upregulated (eg. increased or enhanced; herein referred to as “agonism” of the T cell response) or downregulated (eg. inhibited or retarded; herein referred to as “antagonism” of the T cell response) relative to the nature of the response which would occur in response to the unmodified form of the peptide. Accordingly, reference to “agonism” and “antagonism” are not intended as a reference to the physical agonism or antagonism which can occur between two molecules (such as a T cell receptor and peptide-MHC complex) but are a reference to the nature of the T cell response which is ultimately generated. This is not to say, however, that the agonism or antagonism of the response which is induced is not in fact due to the creation or elimination of a physical agonism or antagonism between the T cell receptor and the MHC-peptide complex as a result of β-amino acid substitution. Preferably, said T cell response is T cell activation.

The present invention therefore preferably provides a method of modulating peptide specific CD8⁺ T cell activation, said method comprising contacting said CD8⁺ T cell with an MHC I-peptide complex, which peptide comprises at least one β-amino acid substitution, wherein said β-amino acid substitution induces either agonism or antagonism of said CD8⁺ T cell activation relative to the CD8⁺ T cell activation inducible by a non-substituted form of said peptide.

Reference to “induces” should be understood as a reference to the direct or indirect induction of the subject modulation. Although it is envisaged that the β-amino acid substitution described herein will generally directly modulate the nature and extent of the signalling which results from T cell receptor interaction with the MHC-peptide complex, it should be understood that in some circumstances the subject modulation may occur indirectly. For example, certain forms of β-amino acid substitution may result in modulation of the stability of the peptide in the MHC cleft, thereby possibly impacting on one or more of the functional activities of the cell which is presenting this peptide, in the context of its role in the T cell response.

The peptide may be derived from any source. For example, it may be derived from a naturally occurring, recombinantly produced or synthetic regenerated polypeptide or protein which, upon uptake or expression within a cell, is subject to processing and presentation of the peptide components derived therefrom in the context of either MHC class I or class II. Alternatively, the peptide may be one which is not derived from a larger molecule, such as a protein or polypeptide, but which in the first instance takes the form of a peptide.

Examples of peptides include but are not limited to, peptides derived from proteinaceous components of microorganisms (such as bacteria, viruses, fungi or parasites), synthetically generated or naturally occurring proteinaceous toxins, environmental proteinaceous antigens or self molecules (such as those against which autoimmune responses are directed). It should be understood that the subject peptide may be immunogenic or non-immunogenic. In terms of non-immunogenic peptides, one of the objects of the present invention is to provide a means of effectively improving immunogenicity by agonising the response of T cells expressing a TCR directed to that peptide, which T cells would not ordinarily undergo immunologically significant activation in response to the naturally occurring form of that peptide.

The peptide which is utilised in accordance with the method of the present invention may take any suitable form. For example, the peptide may be glycosylated or un-glycosylated, phosphorylated or dephosphorylated to various degrees and/or may contain a range of other proteinaceous or non-proteinaceous molecules fused, linked, bound or otherwise associated with the protein such as amino acids, lipids, carbohydrates or other peptides, polypeptides or proteins.

Without limiting the present invention in any way, naturally occurring proteins (and therefore the peptides derived therefrom) are composed of α-amino acids, being the naturally occurring form which amino acids generally take. The present invention is predicated on substituting one or more of the α-amino acids comprising a peptide sequence with a β-amino acid and, preferably, the corresponding β-amino acid.

Preferably the peptide is derived from:
(i) a tumour target such as NY-ESO, MUC1, MAGE, BAGE, RAGE or CAGE family members.
(ii) viral targets such as EBV, CMV, HIV or HCV.
(iii) tolerogenic epitopes such as MBP, which is an antigen in multiple sclerosis.

As used herein, the term “amino acid” refers to an α-amino acid or a β-amino acid and may be a L- or D-isomer. The amino acid may have a naturally occurring side chain (see Table 1 above) or a non-naturally occurring side chain (see Table 2 below). The amino acid may also be further substituted in the α-position or the β-position with a group selected from —C₁-C₁₀alkyl, —C₂-C₁₀alkenyl, —C₂-C₁₀alkynl, —(CH₂)ₙCOR₁, —(CH₂)ₙR₂, —PO₃H, —(CH₂)ₙheterocyclyl or —(CH₂)ₙaryl where R₁ is —OH, —NH₂, —NHC₁-C₃alkyl, —OC₁-C₃alkyl or —C₁-C₃alkyl and R₂ is —OH, —SH, —SC₁-C₃alkyl, —OC₁-C₃alkyl, —C₃-C₂cycloalkyl, —C₃-C₁₂cycloalkenyl, —NH₂, —NHC₁-C₃alkyl or —NHC(C═NH)NH₂, n is 0 or an integer from 1 to 10 and where each alkyl, alkenyl, alkynyl, cycloalkyl, cycloalkenyl, aryl or heterocyclyl group may be substituted with one or more groups selected from —OH, —NH₂, —NHC₁-C₃alkyl, —OC₁-C₃alkyl, —SH, —SC₁—C₃alkyl, —CO₂H, —CO₂C₁-C₃alkyl, —CONH₂ or —CONHC₁-C₃alkyl.

The term “α-amino acid” as used herein, refers to an compound having an amino group and a carboxyl group in which the amino group and the carboxyl group are separated by a single carbon atom, the α-carbon atom. An α-amino acid includes naturally occurring and non-naturally occurring L-amino acids and their D-isomers and derivatives thereof such as salts or derivatives where functional groups are protected by suitable protecting groups. The α-amino acid may also be further substituted in the α-position with a group selected from —C₁-C₁₀alkyl, —C₂-C₁₀alkenyl, —C₂-C₁₀alkynyl, —(CH₂)ₙCOR₁, —(CH₂)ₙR₂, —PO₃H, —(CH₂)ₙheterocyclyl or —(CH₂)ₙaryl where R₁ is —OH, —NH₂, —NHC₁-C₃alkyl, —OC₁-C₃alkyl or —C₁-C₃alkyl and R₂ is —OH, —SH, —SC₁-C₃alkyl, —OC₁-C₃alkyl, —C₃-C₁₂cycloalkyl, —C₃-C₁₂cycloalkenyl, —NH₂, —NHC₁-C₃alkyl or —NHC(C═NH)NH₂, n is 0 or an integer from 1 to 10 and where each alkyl, alkenyl, alkynyl, cycloalkyl, cycloalkenyl, aryl or heterocyclyl group may be substituted with one or more groups selected from —OH, —NH₂, —NHC₁-C₃alkyl, —OC₁-C₃alkyl, —SH, —SC₁-C₃alkyl, —CO₂H, —CO₂C₁-C₃alkyl, —CONH₂ or —CONHC₁-C₃alkyl.

As used herein, the term “β-amino acid” refers to an amino acid that differs from an α-amino acid in that there are two (2) carbon atoms separating the carboxyl terminus and the amino terminus. As such, β-amino acids with a specific side chain can exist as the R or S enantiomers at either of the α (C2) carbon or the β (C3) carbon, resulting in a total of 4 possible isomers for any given side chain. The side chains may be the same as those of naturally occurring α-amino acids (see Table 1 above) or may be the side chains of non-naturally occurring amino acids (see Table 2 below).

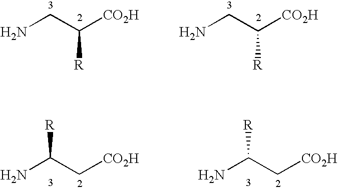

Furthermore, the β-amino acids may have mono-, di-, tri- or tetra-substitution at the C2 and C3 carbon atoms. Mono-substitution may be at the C2 or C3 carbon atom. Di-substitution includes two substituents at the C2 carbon atom, two substituents at the C3 carbon atom or one substituent at each of the C2 and C3 carbon atoms. Tri-substitution includes two substituents at the C2 carbon atom and one substituent at the C3 carbon atom or two substituents at the C3 carbon atom and one substituent at the C2 carbon atom. Tetra-substitution provides for two substituents at the C2 carbon atom and two substituents at the C3 carbon atom. Suitable substituents include —C₁-C₁₀alkyl, C₂-C₁₀alkenyl, C₂-C₁₀alkynyl, —(CH₂)ₙCOR₁, —(CH₂)ₙR₂, —PO₃H, —(CH₂)ₙheterocyclyl or —(CH₂)ₙaryl where R₁ is —OH, —NH₂, —NHC₁-C₃alkyl, —OC₁-C₃alkyl or —C₁-C₃alkyl and R₂ is —OH, —SH, —SC₁-C₃alkyl, —OC₁-C₃alkyl, —C₃-C₁₂cycloalkyl, —C₃-C₁₂cycloalkenyl, —NH₂, —NHC₁-C₃alkyl or —NHC(C═NH)NH₂ and where each alkyl, alkenyl, alkynyl, cycloalkyl, cycloalkenyl, aryl or heterocyclyl group may be substituted with one or more groups selected from —OH, —NH₂, —NHC₁-C₃alkyl, —OC₁-C₃alkyl, —SH, —SC₁-C₃alkyl, —CO₂H, —CO₂C₁-C₃alkyl, —CONH₂ or —CONHC₁-C₃alkyl.

Other suitable β-amino acids include conformationally constrained β-amino acids. Cyclic β-amino acids are conformationally constrained and are generally not accessible to enzymatic degradation. Suitable cyclic β-amino acids include, but are not limited to, cis and trans 2-amino-C₃-C₁₀-cycloalkyl-1-carboxylic acids, 2-amino-C₃-C₁₀-cycloalkenyl-1-carboxylic acids, 2-amino-norborane-1-carboxylic acids and their unsaturated carboxylic acid examples of suitable conformationally constrained β-amino acids include 2-aminocyclopropyl carboxylic acids, 2-aminocyclobutyl and cyclobutenyl carboxylic acids, 2-aminocyclopentyl and cyclopentenyl carboxylic acids, 2-aminocyclohexyl and cyclohexenyl carboxylic acids and 2-amino-norbornane carboxylic acids and tropane amino acids and their derivatives, some of which are shown below:

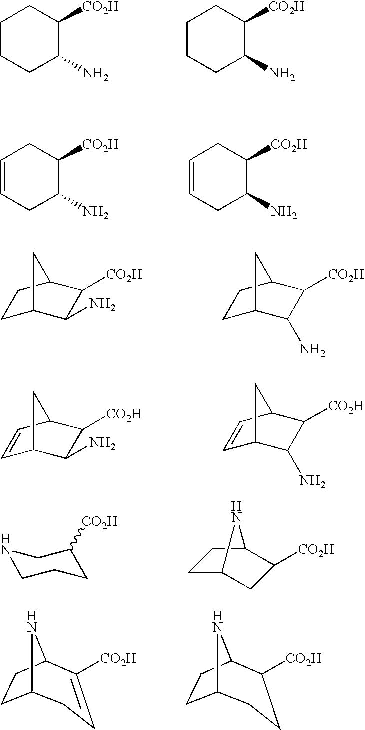

Suitable derivatives of β-amino acids include salts and may have functional groups protected by suitable protecting groups.

The term “non-naturally occurring amino acid” as used herein, refers to amino acids having a side chain that does not occur in the naturally occurring L-α-amino acids. Examples of non-natural amino acids and derivatives include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids that may be useful herein is shown in Table 2.

TABLE 2

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyl-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

The term “alkyl” as used herein refers to straight chain or branched hydrocarbon groups. Suitable alkyl groups include, but are not limited to methyl, ethyl, propyl, isopropyl, butyl, isobutyl, pentyl, hexyl, heptyl, octyl, nonyl and decyl. For example, C₁-C₃alkyl refers to methyl, ethyl, propyl and isopropyl.

The term “alkenyl” as used herein refers to straight chain or branched unsaturated hydrocarbon groups containing one or more double bonds. Suitable alkenyl groups include, but are not limited to ethenyl, propenyl, isopropenyl, butenyl, pentenyl, hexenyl, heptenyl, octenyl, nonenyl and decenyl.

The term “alkynyl” as used herein refers to straight chain or branched unsaturated hydrocarbon groups containing one or more triple bonds. Suitable alkynyl groups include, but are not limited to ethynyl, propynyl, butynyl, pentynyl, hexynyl, heptynyl, octynyl, nonynyl and decynyl.

The term “cycloalkyl” as used herein, refers to cyclic hydrocarbon groups. Suitable cycloalkyl groups include, but are not limited to, cyclopropyl, cyclobutyl, cyclopentyl, cyclohexyl, cycloheptyl, cyclooctyl, cyclononyl, cyclodecyl, cycloundecyl and cyclododecyl.

The term “cycloalkenyl” as used herein, refers to cyclic unsaturated hydrocarbon groups having at least one double bond in the ring. Suitable cycloalkenyl groups include, but are not limited to cyclopropenyl, cyclobutenyl, cyclopentenyl, cyclohexenyl, cycloheptenyl, cyclooctenyl, cyclononenyl, cyclodecenyl, cycloundecenyl and cyclododecenyl.

The term “heterocyclyl” as used herein refers to 5 or 6 membered cyclic hydrocarbon groups in which at least one carbon atom has been replaced by N, O or S. Optionally, the heterocyclyl group may be fused to a phenyl ring. Suitable heterocyclyl groups include, but are not limited to pyrrolidinyl, piperidinyl, pyrrolyl, thiophenyl, furanyl, oxazolyl, imidazolyl, thiazolyl, isoxazolyl, pyrazolyl, isothiazolyl, pyridinyl, quinolinyl, isoquinolinyl, indolyl, benzofuranyl, benzothiophenyl, oxadiazolyl, tetrazolyl, triazolyl and pyrimidinyl.

The term “aryl” as used herein, refers to C₆-C₁₀ aromatic hydrocarbon groups, for example phenyl and naphthyl.

It will also be recognised that the compounds of formula (I) possess asymmetric centres and are therefore capable of existing in more than one stereoisomeric form. The invention thus also relates to compounds in substantially pure isomeric form at one or more asymmetric centres eg., greater than about 90% ee, such as about 95% or 97% ee or greater than 99% ee, as well as mixtures, including raceinic mixtures, thereof. Such isomers may be naturally occurring or may be prepared by asymmetric synthesis, for example using chiral intermediates, or by chiral resolution.

As detailed hereinbefore, the method for the present invention is predicated on replacing the α form of any one or more amino acids of the peptide of interest with the corresponding β form of that amino acid. Accordingly, reference to “substituting” should be understood as a reference to this replacement event. However, it should be understood that although it is preferable that a given α-amino acid be replaced with the β form of that particular amino acid, it may be possible to achieve the same functional outcome by conservatively substituting the subject α-amino acid with a different amino acid, albeit in β-amino acid form. Methods of determining suitable conservative substitutions would be well known to those of skill in the art and could be performed as a matter of routine procedure.

Typical conservative amino acid substitutions include, but are not limited to, those detailed in Table 3, below:

TABLE 3
Suitable residues for amino acid substitutions

| Original α-amino | Exemplary β-amino |
|---|---|
| acid Residue | acid Substitutions |
| Ala | Ser |
| Arg | Lys |
| Asn | Gln; His |
| Asp | Glu |
| Cys | Ser |
| Gln | Asn |
| Glu | Ala |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Any number of the α-amino acids comprising a peptide of interest may be substituted by the corresponding β-amino acid. As few as one amino acid or up to all subject amino acids may be substituted, although it is anticipated that the number of substitution events will lie midway between these figures. In light of the teachings provided herein (both in terms of the means of generating a panel of substituted peptides for testing and the in vitro testing methodology described herein), the person of skill in the art could now, as a matter of routine procedure, determine the nature of the substitutions which are required to be made in order to achieve the objectives of the present invention. For example, most of the common MHC alleles are now known and have been the subject of x-ray crystallographic analysis. Accordingly, identifying potential anchor positions and effecting substitutions thereof can be routinely elucidated. Even to the extent that some MHC alleles are not currently known in terms of their three-dimensional structure, the technique of pool sequencing provides a routine method of nevertheless identifying the MHC anchoring positions of a peptide of interest. Pool sequencing is a well known technique which provides for the analysis of sequence of a number of epitopes which bind a given MHC allele thereby enabling the identification of consensus motifs. In terms of determining the form of MHC molecule which a given epitope binds, there are standard methods known to those of skill in the art which provide for the identification of the particular MHC restriction molecule, such as a particular HLA restriction molecule, to which an epitope binds. These methods similarly do not rely on the knowledge of three-dimensional MHC structures. In terms of the analysis of T cell responsiveness, the person of skill in the art may either generate appropriate T cell clones for a given epitope of interest or, alternatively, where an epitope has been shown to generate an immune response in a subject, isolate a cell population such as the peripheral blood mononuclear cells in order to analyse immune cell responsiveness, such as cytotoxic T cell responsiveness. In this regard, it should be understood that although the present invention is exemplified utilising an ovalbumin peptide-MHC I model, this is not intended as a limitation on the broad application of this invention. Rather, the teachings provided herein now facilitate the application of this invention in relation to any peptide which is presentable to T cells by MHC and, in particular, MHC I presented peptides.

Without limiting the present invention to any one theory or mode of action, the inventors have determined that substituting the MHC anchor residues of a given peptide is a particularly preferred means of modulating a T cell response. In this regard, it is thought that although the MHC anchor residues do not directly interact with the T cell receptor, substitution of these residues with their β amino acid counterparts results in a certain degree of conformational change which is thought to then impact on the T cell receptor related signalling. Further, in another preferred embodiment, the subject residues which are substituted in a given peptide of interest correspond to the solvent exposed residues.

However, in light of the teachings and principles provided herein, means for determining and designing appropriate peptides to achieve the object of the present invention would be a matter of routine procedure and include, but are not limited to, application of the following assays (which test vaccine efficacy and T cell recognition):
(i) the use of T cell clones or lines, T cell hybridomas and cytokine production as a read out of TcR ligation and T cell activation. Similar assays exist for immune lymphocytes for spleen or lymph node. Cytokine production, e.g. IL-1, IFN-gamma, TNF-alpha, MIP, RANTES) can be assessed by intracellular cytokine staining (ICS), ELISA assays on co-culture supernatants, protein micro-arrays, ELISpot, or using a bioassay, such as IL-2 sensitive cell lines CTLL.
(ii) Lymphocyte activation can also be assessed by proliferation using uptake of radiolabel (e.g. 3H-thymidine), or chromophores such as BrdU.
(iii) In vivo activity and antigen presentation to lymphocytes can also be measured by in vivo CTL assays or proliferation of adoptively transferred CFSE labeled T cells (Lyons-Parish analysis).
(iv) Humanised responses (i.e. HLA A2-restricted responses for example) can be examined using HLA transgenic mice with the assays listed above or by in vitro stimulation of human PBMCs (using monocyte derived dendritic cellss as APC for instance).

The β-amino acid peptides of the present invention may be prepared by using the methods depicted or described herein or known in the art. It will be understood that minor modifications to methods described herein or known in the art may be required to synthesize particular b-amino acid compounds. General synthetic procedures applicable to synthesis of compounds may be found in standard references such as Comprehensive Organic Transformations, R. C. Larock, 1989, VCH publishers and Advanced Organic Chemistry, J. March, 4^{th} Edition (1992), Wiley InterScience, and references therein. It will also be recognised that certain reactive groups may require protection and deprotection during the synthetic process preparing β-amino acids or peptides containing them.

Suitable protecting and deprotecting methods for reactive functional groups are known in the art, for example in Protective Groups in Organic Synthesis, T. W. Green & P. Wutz, John Wiley & Son, 3^{rd} Edition, 1999 and Amino Acid and Peptide Synthesis, John Jones, Oxford Science Publications, 1992.

β-amino acids having the R side chain group at C2 may be prepared with the exemplified general method depicted in Scheme 1. Suitable starting materials can be obtained commercially or prepared using methods known in the art.

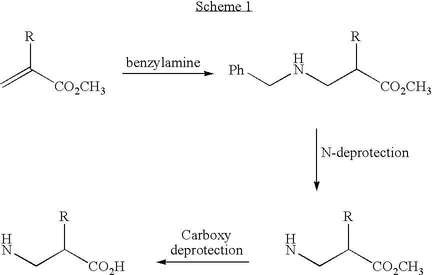

β-amino acids having the R side chain group at C3 may be prepared with the exemplified general method depicted in Scheme 2. Suitable starting materials can be obtained commercially or prepared using methods known in the art.

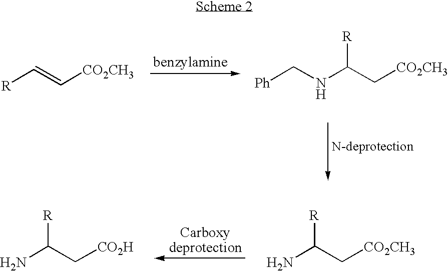

Alternatively, β-amino acids having the R side chain group at C3 may be prepared with the exemplified general method depicted in Scheme 3 using the Arndt-Eistert reaction. Suitable starting materials can be obtained commercially, for example, α-amino acids or prepared using methods known in the art.

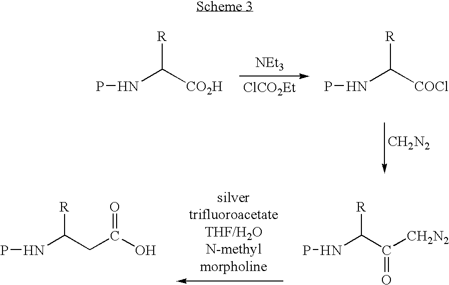

Peptides containing the β-amino acids may be synthesized using known solution peptide synthesis techniques or solid phase techniques. A typical solid phase synthesis is shown in Scheme 4.

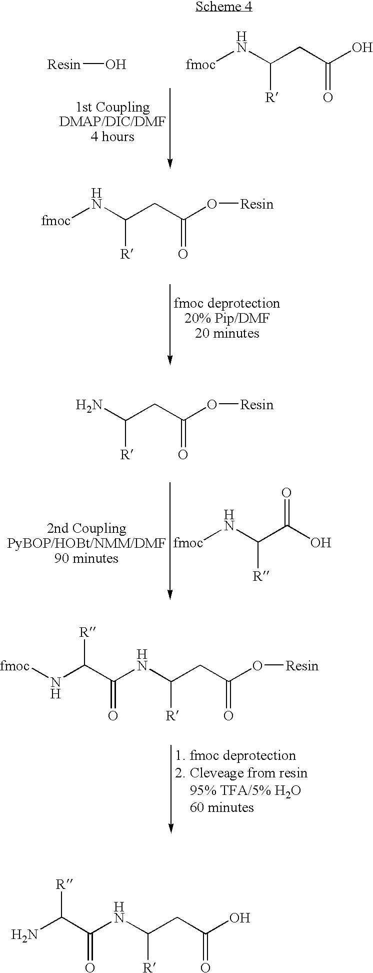

The peptides of the present invention may also optionally undergo any one or more general modifications, in addition to β-amino acid substitution, for any suitable reason such as, but not limited to, increasing solubility of the peptide or increasing its resistance to proteolytic degradation. The peptides may also be modified to incorporate one or more polymorphisms resulting from natural allelic variation, D-amino acids, non-natural amino acids or amino acid analogues. Reporter groups may also be added to facilitate purification and potentially increase solubility of the peptides according to the invention. Other well known types of modifications including insertion of specific endoprotease cleavage sites, addition of functional groups or replacement of hydrophobic residues with less hydrophobic residues. The various modifications to peptides according to the invention which have been mentioned above are mentioned by way of example only and are merely intended to be indicative of the broad range of modifications which can be effected in relation to the subject peptides. In this regard, for example, reference to an “α-amino acid”, per se, should be understood to encompass non β-amino acid substitutes and derivatives of that α-amino acid such as naturally and non-naturally occurring α-amino acid substitutions and analogues detailed in Tables 1, 2 and 3.

Without limiting the present invention to any one theory or mode of action, cytotoxic T cells recognise peptide antigens presented by target cell MHC class I molecules through their T cell receptor. The T cell receptor/peptide-MHC interface is the focal point of the immune synapse. Antigenic signals generally beginning from a viral or tumor protein result in the activation of killing pathways, cytokine production, serine esterase release, calcium influx and cell proliferation that ultimately removes the infected/damaged cells. A T cell receptor only reads a very small portion of the presented peptide. In the context of the peptide exemplified herein, being ovalbumin, the ovalbumin peptide composition at Asn-P4, Glu-P6 and Lys-P7 is sensitive to substitution and results in dramatic changes to T cell receptor signalling when modified. In this regard, it was determined that particular β-amino acid substitutions led to a generalised agonism of the T cell response whether the substituted amino acids were solvent exposed or not.

The present invention therefore provides a means of modulating the nature of the T cell response to a peptide of interest. This provides extensive potential in the context of immunotherapeutics and the development of peptide vaccines and includes, but is not limited to, the administration of modified peptides which can:
(i) Upregulate or otherwise augment the T cell response to antigens which in their natural state exhibit little or no immunogenicity, such as the weak immunogenicity of peptide epitopes derived from tissue antigens expressed by tumors.
(ii) Induce the induction of anergy or tolerance of pathological T cells in a highly efficient and specific manner without compromising the general functioning of the immune system. Such a regime has application in the treatment of any unwanted immune response, such as those associated with autoimmunity, transplant rejection and allergies. In terms of these applications, the advent of the present invention provides a viable alternative to the currently utilised immunosuppressant regimes.
(iii) Provide adjuvant like properties by stabilising the presenting MHC molecule thereby facilitating longer presentation at the cell surface. This provides a more intense and longer lasting immunological effect.
(iv) Induce a polyclonal T cell response that may utilize alternate TcR Vα and Vβ elements relative to those induced by vaccination with the all α-amino acid containing peptide. This has significant potential in chronic infections where T cell specificities may become exhausted and T cells of a given specificity may be difficult to elicit or in situations where the target response is towards an self-antigen that has been the subject of tolerance induction, e.g. many tumour antigens fall in this category of self antigens.
(v) Provide for stabilisation of a peptide in the immunisation vehicle. For example, a number of peptides are susceptible to proteolysis and others forms of degradation and are therefore difficult to formulate for administration to an individual, such as via intravenous means, due to their instability. Accordingly, stabilising these peptides according to the method of the present invention provides a means of increasing their bioavailability thereby rendering these peptide vaccines more effective.

Accordingly, yet another aspect of the present invention provides a method of modulating a peptide specific T cell response in a subject, said method comprising administering to said subject an effective amount of a peptide, which peptide comprises at least one β-amino acid substitution, for a time and under conditions sufficient to present said peptide to said T cell in the context of an MHC-peptide complex, wherein said β-amino acid substitution induces either agonism or antagonism of said T cell response relative to the T cell response inducible by a non-substituted form of said peptide.

More particularly, the present invention provides a method of modulating a peptide specific cytotoxic T cell response in a subject, said method comprising administering to said subject an effective amount of a peptide, which peptide comprises at least one β-amino acid substitution, for a time and under conditions sufficient to present said peptide to said cytotoxic T cell in the context of an MHC I-peptide complex, wherein said β-amino acid substitution induces either agonism or antagonism of said cytotoxic T cell response relative to the cytotoxic T cell response inducible by a non-substituted form of said peptide.

Preferably, said T cell response is T cell activation.

The subject of the treatment or prophylaxis is generally a mammal such as but not limited to human, primate, livestock animal (e.g. sheep, cow, horse, donkey, pig), companion animal (e.g. dog, cat), laboratory test animal (e.g. mouse, rabbit, rat, guinea pig, hamster), captive wild animal (e.g. fox, deer). Preferably the mammal is a human or primate. Most preferably the mammal is a human.

In yet another aspect, the inventors have surprisingly determined that a peptide which is capable of inducing either agonism or antagonism of the T cell response of an isolated T cell clone can, in fact, induce augmentation of the opposite response (ie. antagonism or agonism, respectively) when administered together with the unsubstituted form of the peptide, and when considered relative to the degree of response which is in fact inducible by the unsubstituted peptide alone.

Accordingly, yet another aspect of the present invention provides a means of agonising a peptide specific T cell response in a subject, said method comprising co-administering to said subject an effective amount of an agonistic peptide, which peptide comprises at least one β-amino acid substitution, together with the non-substituted form of said peptide for a time and under conditions sufficient to present said peptides to said T cells in the context of an MHC-peptide complex.

In yet another aspect there is provided the method of antagonising a peptide specific T cell response in a subject, said method comprising co-administering to said subject an effective amount of an agonistic peptide, which peptide comprises at least one β-amino acid substitution, together with the non-substituted form of said peptide for a time and under conditions sufficient to present said peptides to said T cells in the context of an MHC-peptide complex.

Reference to “antagonistic” peptide should be understood as a reference to a β-amino acid substituted peptide which induces antagonism of the T cell response of an isolated T cell relative to the T cell response inducible by a non-substituted form of said peptide. Reference to “agonistic” peptide should be understood as a reference to a β-amino acid substituted peptide which induces agonism of the T cell response of an isolated T cell relative to the T cell response inducible by a non-substituted form of said peptide. In light of the teachings provided herein, it would now be a matter of routine procedure for the person of skill in the art to determine whether any given β-amino acid substituted peptide is suitable for use in accordance with these aspects of the present invention. For example, methods for routinely determining the antagonistic or agonistic properties of the given peptides in the context of an isolated T cell clone are well known. Further, methods for determining the overall cumulative response of a polyclonal T cell population in the presence of both the substituted and non-substituted forms of the peptide could also be performed as a matter of routine procedure, thereby providing a routine means of designing prophylactic and therapeutic methodology based on inducing this previously undescribed phenomenon.

Reference to “co-administered” should be understood to encompass the simultaneous administration in the same formulation or in two different formulations of the subject peptides via the same or different routes or their sequential administration by the same or different routes. By “sequential administration” is meant a time difference of seconds, minutes, hours or days between the administration of the two types of molecules. These molecules may be administered in any order. “Co-administration” should also be understood to encompass the administration of one peptide where the other peptide is already present in the subject. For example, one may seek to administer a β-amino acid substituted peptide which corresponds to a naturally occurring self-peptide, which self-peptide need not be administered, per se, since this peptide is naturally present in an individual. This may occur, for example, in the context of treating autoimmune diseases.

As detailed hereinbefore, the present invention should also be understood to extend to the use of the method of the present invention in the therapeutic and/or prophylactic treatment of patients, such as the treatment and/or prophylaxis of disease conditions or other unwanted conditions.

The present invention therefore contemplates a method for the treatment and/or prophylaxis of a condition characterised by an aberrant, unwanted or otherwise inappropriate peptide specific T cell response in a subject, said method comprising administering to said subject an effective amount of a peptide, which peptide comprises at least one β-amino acid substitution, for a time and under conditions sufficient to present said peptide to said T cell in the context of an MHC-peptide complex, wherein said β-amino acid substitution induces either agonism or antagonism of said T cell response relative to the T cell response inducible by a non-substituted form of said peptide.

Preferably said T cell is a cytotoxic T cell and said MHC-peptide complex is an MHC I-peptide complex. Still more preferably said T cell response is T cell activation.

Reference to an “aberrant, unwanted or otherwise inappropriate” T cell response should be understood as a reference to an underactive response, to a physiologically normal response which is inappropriate in that it is unwanted or to an overactive response. As detailed hereinbefore, there are many conditions which are dependent on the induction of the correct level and nature of a T cell response. For instance, an allergic response is technically an immunologically normal response, but which response is nevertheless unwanted and unnecessary in the context of an innocuous antigen. In another example, a T cell response directed to self antigens results in the induction of autoimmune conditions which are highly undesirable. In yet another example, the highly inefficient T cell responses which are sometimes induced to very weakly immunogenic peptides can result in fatal conditions where that peptide is derived from a highly pathogenic organism or tumor. Accordingly, the method of the present invention has extensive scope in relation to such conditions.

In a preferred embodiment, where the subject aberrant, unwanted or otherwise inappropriate T cell response is an inadequate T cell response, preferably said β-amino acid substituted peptide induces agonism of the subject response relative to that inducible by a non-substituted form of said peptide. In another example, where said aberrant, unwanted or otherwise inappropriate T cell response is an overactive response or even the mere occurrence of any degree of response, said β-amino acid substituted peptide induces antagonism of said response relative to the response inducible by a non-substituted form of said peptide.

Another aspect of the present invention provides a method for the treatment and/or prophylaxis of a condition characterised by the occurrence of an unwanted peptide specific T cell response in a subject, said method comprising co-administering to said subject an effective amount of an agonistic peptide, which peptide comprises at least one β-amino acid substitution together with a non-substituted form of said peptide for a time and under conditions sufficient to present said peptides to said T cells in the context of an MHC peptide complex.

In still another aspect there is provided a method for the treatment and/or prophylaxis of a condition characterised by an inadequate peptide specific T cell response in a subject, said method comprising co-administering to said subject an effective amount of an antagonist peptide, which peptide comprises at least one β-amino acid substitution, together with a non-substituted form of said peptide for a time and under conditions sufficient to present said peptides to said T cells in the context of an MHC peptide complex.

Preferably, said condition characterised by an unwanted T cell response is an autoimmune condition, a transplant or an allergic condition. Preferably said condition characterised by an inadequate T cell response is a neoplastic condition or an infection.

An “effective amount” means an amount necessary at least partly to attain the desired response, or to delay the onset or inhibit progression or halt altogether, the onset or progression of the particular condition being treated. The amount varies depending upon the health and physical condition of the individual to be treated, the taxonomic group of the individual to be treated, the degree of protection desired, the formulation of the composition, the assessment of the medical situation, and other relevant factors. It is expected that the amount will fall in a relatively broad range that can be determined through routine trials.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

The present invention further contemplates a combination of therapies, such as the administration of the modulatory agent together with other proteinaceous or non-proteinaceous molecules which may facilitate the desired therapeutic or prophylactic outcome.

Administration of the peptides of the present invention hereinbefore described [herein collectively referred to as “modulatory agent”], in the form of a pharmaceutical composition, may be performed by any convenient means. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 mg to about 1 mg of modulatory agent may be administered per kilogram of body weight per day. Dosage regimes may be adjusted to provide the optimum therapeutic response. For example, several divided doses may be administered daily, weeldy, monthly or other suitable time intervals or the dose may be proportionally reduced as indicated by the exigencies of the situation.

The modulatory agent may be administered in a convenient manner such as by the oral, intravenous (where water soluble), intraperitoneal, intramuscular, subcutaneous, intradermal or suppository routes or implanting (e.g. using slow release molecules). The modulatory agent may be administered in the form of pharmaceutically acceptable non-toxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant. Preferably, said route of administration is subcutaneous or oral.

In still another aspect, the present invention contemplates the use of a β-amino acid substituted peptide as hereinbefore defined in the manufacture of a medicament for the treatment of a condition in a mammal, which condition is characterised by an aberrant, unwanted or otherwise inappropriate T cell response wherein said β-amino acid substitution induces either agonism or antagonism of said T cell response relative to the T cell response inducible by a non-substituted from of said peptide.

Preferably said T cell response is a cytotoxic T cell response and said MHC peptide complex is an MHC-I peptide complex. Even more preferably said T cell response is T cell activation.

The method of the present invention contemplates the modulation of T cell functioning both in vitro and in vivo. Although the preferred method is to treat an individual in vivo it should nevertheless be understood that it may be desirable that the method of the invention may be applied in an in vitro environment, for example to induce the activation of virgin T cells for purposes such as the creation of T cell lines directed to poorly immunogenic peptides.

In yet another further aspect, the present invention contemplates a pharmaceutical composition comprising the modulatory agent as hereinbefore defined and one or more pharmaceutically acceptable carriers and/or diluents. Said agents are referred to as the active ingredients.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding an unsubstituted form of said peptide. The vector may, for example, be a viral vector.

It should also be understood that in addition to administering the peptides of the present invention, per se, the present invention is also directed to administering the subject peptides together with any proteinaceous or non-proteinaceous molecule such as molecules which may assist in targeting the peptide to primary or secondary lymphoid organs. Further, it should be understood that the method of the present invention also extends to the administration of peptide which has already been expressed in the context of MHC, such as the administration of MHC expressing cells which have already been loaded with antigens.

In yet another aspect, there has been developed a method of designing and screening for β-amino acid substituted peptide analogues, which method provides a means of rationally substituting α-amino acids for β-amino acids in a positional scanning approach and the identification of peptides exhibiting desired functional activity and improved bioavailability. The subject method is based on generating a population of peptide analogues by the sequential substitution of one or more of the α-amino acids comprising the peptide of interest with the corresponding β-amino acid and functionally analysing the. analogues derived therefrom. Accordingly, it should be understood that the present invention extends to this screening method and the peptides derived therefrom.

The present invention is further described by the following non-limiting figures and/or examples.

### EXAMPLE 1

### Modification of MHC Class I Binding and CTL Response By B-Amino Acid Substituted Peptides

### Methods and Materials

Cell Lines

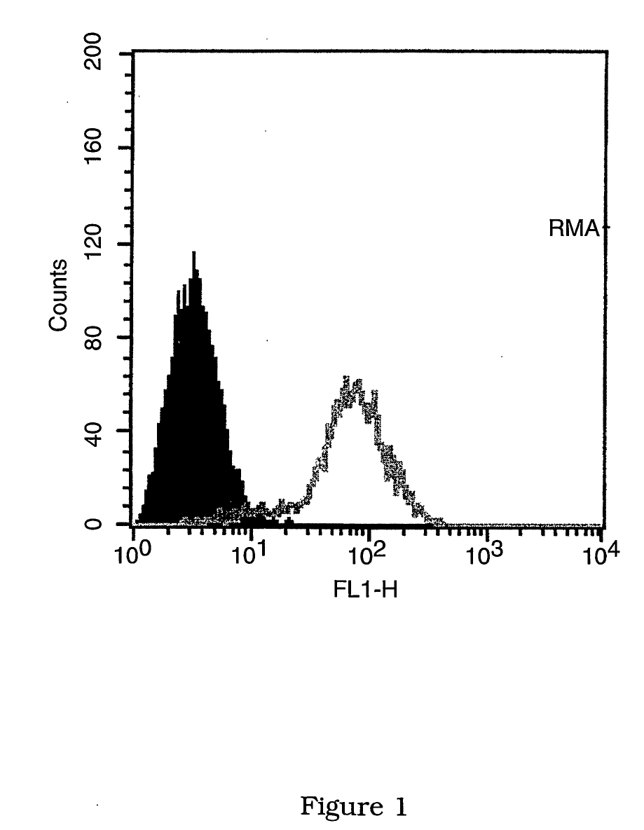

RMA-S cells expressing the MHC Class I H2-K^{b} (Schumacher, N. M., M.-T. Heemels, J. J. Neefjes, W. M. Kast, C. J. Melief, M. H. L. Ploegh. 1990. Direct binding of peptide to empty MHC class I molecules on intact cells and in vitro. Cell 62:563; Karre K., Ljunggren H. G., Piontek G., and Kiessling R. Selective rejection of H-2-deficient lymphoma variants suggests alternative immune defence strategy. Nature. Feb. 20-26, 1986; 319(6055):675-8) were kept in Dulbecco's Modified Eagles Medium (DMEM) media containing 10% fetal calf serum (FCS), L-glutamine and antibiotics (0.5 mg/ml G418). These cells express low levels of K^{b} (˜5% -10% of normal) on the cell surface at 37° C., due to a mutation in the TAP (transporter associated with antigen processing) molecule of these cells. Incubation of RMA-S cells at temperatures between 19° and 33° C. results in an amplification of the surface expression of K^{b} (˜50% present in the RMA parental cell line)²⁰. These molecules are bound by sub-optimal peptide ligands and as a result are unstable when these cells are returned to 37° C. In fact only when high affinity exogenous peptides are added to the RMA-S cells will this amplification in K^{b} expression be observed. This makes these cells an ideal whole cell reporter assay for ligand binding to K^{b} molecules, and as shown in FIG. 1, exogenous peptide results in a 50-fold increase in surface K^{b} staining as revealed by flow cytometry.

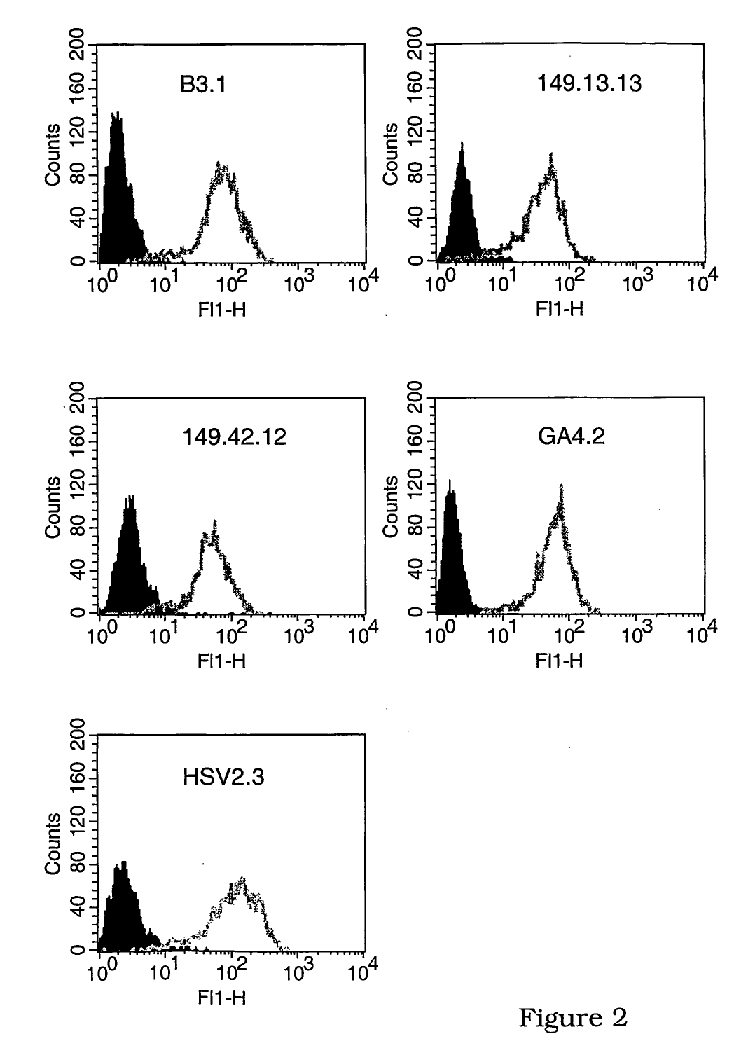

The SIINFEKL/K^{b}-restricted CD8⁺ CTL clones B3.1, GA4.2 (Nikolic-Zugic, J., and Carbone F. R. (1990). The effect of mutations in the MHC Class I peptide binding groove on the cytotoxic T lymphocyte recognition of the Kb restricted ovalbumin determinant. Eur. J. Immunol. 20:2431), 149.13.13, 149.42.12 (Clark S. R., Barnden M., Kurts C., Carbone F. R., Miller J. F., Heath J. R. Characterization of the ovalbumin-specific TCR transgenic line OT-I: MHC elements for positive and negative selection. Immunol Cell Biol. April 2000;78(2): 110-7), and the herpes simplex viral specific clone HSV2.3 (Wallace, M. E., Keating, R., Heath, W R., Carbone, F. R. 1999. The cytotoxic T-cell response to herpes simplex virus type 1 infection of C57BL/6 mice is almost entirely directed against a single immunodominant determinant. J Virol. September;73(9):7619-26) were derived and maintained as described. All T cell hybrids were grown in commercial DMEM containing 10% fetal calf serum, L-glutamine and antibiotics (0.7 mg/ml G418). CTL clones were used in recognition assays, and clones B3.1 and GA4.2 were used in antagonism assays. T hybrids were extensively phenotyped prior to use, including examination of T cell function and cell surface phenotype (flow cytometric staining for CD8; see FIG. 2).

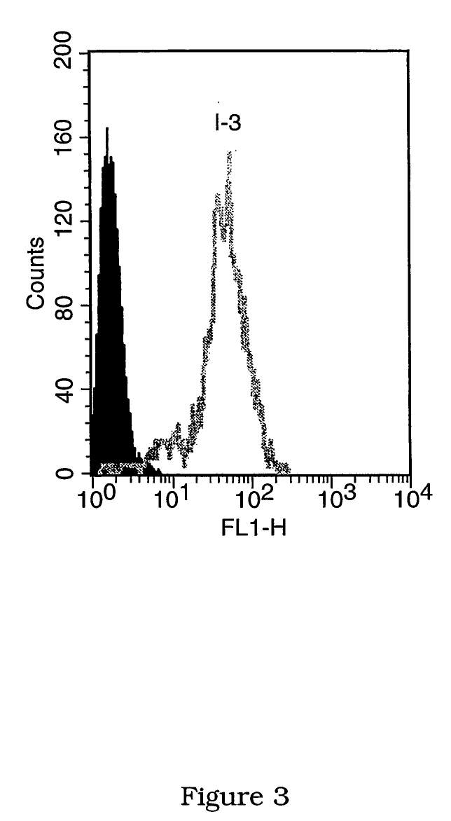

I-3 fibroblast cells which express MHC Class I H2-K^{b} (see FIG. 3) were grown in DMEM-10. I-3 cells were used as target cells in CTL recognition and antagonism assays.

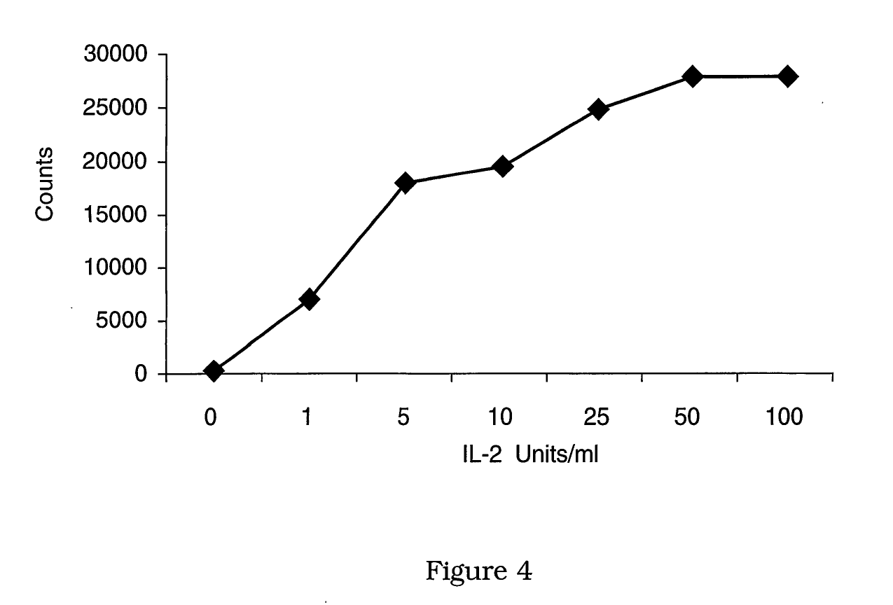

The interleukin 2 (IL-2) dependent cell line, CTLL-2 (Gillis, S., M. M. Ferm, W. Ou, K. A. Smith. 1978. T cell growth factor: parameters of production and a quantitative microassay for activity. J. Immunol. 120:2027), were used in an IL-2 bioassay to determine the relative amount of IL-2 production by CTL clones as stimulated by peptide analogues (Chang, H. C., A. Smolyar, R. Spoerl, T. Witte, Y. Yao, E. C. Goyarts, S. G. Nathenson, E. L. Reinherz. 1997. Topology of T cell receptor-peptide/class I MHC interaction defined by charge reversal complementation and functional analysis. J. Mol. Biol. 271:278). Cells were grown in DMEM-10 supplemented with 100 U/ml rIL-2 and upon removal of IL-2 in the culture medium CTLL proliferated in a dose dependent manner to exogenous IL-2 (see FIG. 4).

Peptides

Protected β-amino acids were synthesised in collaboration with Dr. Patrick Perlmutter at the Chemistry Department, Monash University. Peptides were synthesised (by Karen Stewart) using solid phase peptide synthesis and the standard f-moc (N-(9-fluorenyl)methoxycarbonyl) protecting strategy. After synthesis and selective deprotection, the complete deprotection and cleavage from the resin was achieved using trifluoroacetic acid (TFA). Purification of peptides was achieved by reverse phase high performance liquid chromatography (RP-HPLC) and purified peptides were characterized by Electrospray ionisation (ESI) Mass spectrometry, and subsequent MS/MS (Department of Microbiology and Immunology, University of Melbourne) using an agilent Technologies LCD ion trap mass spectrometer. This combination of RP-HPLC purification and in particular ion trap MS based experiments allowed unambiguous structural confirmation of each β-analogue. Peptides and their relative molecular mass and purity can be viewed in Table 4.

TABLE 4

The sequence of each peptide, relative molecular mass purity.
Sequence

| | SEQ | | | | | | | | | | |
|---|---|---|---|---|---|---|---|---|---|---|---|
| | ID | | | | | | | | | | |
| Peptide | NO. | P1 | P2 | P3 | P4 | P5 | P6 | P7 | P8 | MW | Purity |
| 1 | 1 | Ser | Ile | Ile | Asn | Phe | Glu | Lys | Leu | 962.8 | >70% |
| 2 | 2 | β^{c3}Ser | Ile | Ile | Asn | Phe | Glu | Lys | Leu | 976.8 | 98% |
| 3 | 3 | Ser | β^{c3}Ile | Ile | Asn | Phe | Glu | Lys | Leu | 976.8 | >80% |
| 4 | 4 | Ser | Ile | β^{c3}Ile | Asn | Phe | Glu | Lys | Leu | 976.8 | 98% |
| 5 | 5 | Ser | Ile | Ile | β^{c3}Asn | Phe | Glu | Lys | Leu | 976.8 | 86% |
| 6 | 6 | Ser | Ile | Ile | Asn | β^{c3}Phe | Glu | Lys | Leu | 976.8 | 85% |
| 7 | 7 | Ser | Ile | Ile | Asn | Phe | β^{c3}Glu | Lys | Leu | 976.8 | 95% |
| 8 | 8 | Ser | Ile | Ile | Asn | Phe | Glu | Lys | β^{c3}Leu | 976.8 | >80% |

H2-K^{b} Stabilisation Assay

RMA-S cells were grown to a density of ˜10⁶ cells/ml at 37° C., then maintained at 26° C. for 20 hrs with 5% CO₂. Cells were washed and resuspended to give a final concentration of 10⁵ cells per well (200 μl ) of a flat-bottom 96-well plate. Cells were pulsed with peptide titrations in PBS, incubated for 1 hr at 26° C. and then an additional 2 hrs at 37° C. This second incubation removes unstable Kb-peptide complexes from the cell surface. Following washes, cell surface expression of H2-K^{b} was detected using the monoclonal antibody Y3 which stains properly conformed Kb complexes on the cell surface (Jones, B., Jr C. A. Janeway. 1981. Cooperative interaction of B lymphocytes with antigen-specific helper T lymphocytes is MHC restricted. Nature 292:547). Following additional washes, Y3 bound Kb-peptide complexes were detected with FITC-conjugated sheep anti-mouse Ig (Amrad Melbourne) during flow cytometry performed on a FACscan (BD Biosciences San Jose Calif.).

SIINFEKL/H2-Kb Stabilisation Assay

The procedure from the K^{b} stabilisation assay above was repeated with the monoclonal antibody 25-D1-16 that specifically binds to SIINFEKL/H2-K^{b} complexes and not to other H2-K^{b} peptide combinations (Porgador, A., J. W. Yewdell, Y. Deng, J. R. Bennink, R. N. Germain. 1997. Localization, quantitation, and in situ detection of specific peptide-MHC class I complexes using a monoclonal antibody. Immunity 6:715).

T-Hybrid Assay

I-3 cells were grown to ˜70% confluent, then plated out into flat-bottom 96-well plate at a concentration of 5×10⁴ cells per well (in a total volume of 100 μl) and incubated at 37° C. for 20 hrs. Cells were then pulsed with peptide titrations for 1 hr at 37° C. T-hybrid clones grown to ˜10⁶ cells/ml were washed and resuspended at a density of 10⁶ cells per ml, then 100 μl transferred to wells containing the pulsed I-3 cells. T-hybrid clones were then incubated in co-culture for 20-24 hrs. 50 μl of co-culture supernatant was harvested from each well and added to the CTLL-2 IL-2 bioassay.

CTLL-2 Bioassay

CTLL-2 cells were grown to ˜5×10⁵ cell per ml then washed 3 times in media containing no exogenous IL-2. Cells were then plated out in a 96 flat-bottom well at a concentration of 5000 cells per well to a final volume of 150 μl . Harvested co-culture supernatant (50 μl) was then added to plated CTLL-2 cells and incubated at 37° C. for 18-22 hrs (depending on visual inspection of control wells with no added IL-2 for CTLL-2 cell death ). Cells were then pulsed with ³H-Thymidine at 1 μCi per well for 6 hrs. Cells were then harvested onto a glass filter and allowed to dry. Once dry, scintillation fluid (Packard Biosciences, Melbourne) was added, then counted on a TopCount scintillation counter (Packard Biosciences, Melbourne).

Antagonism Assay

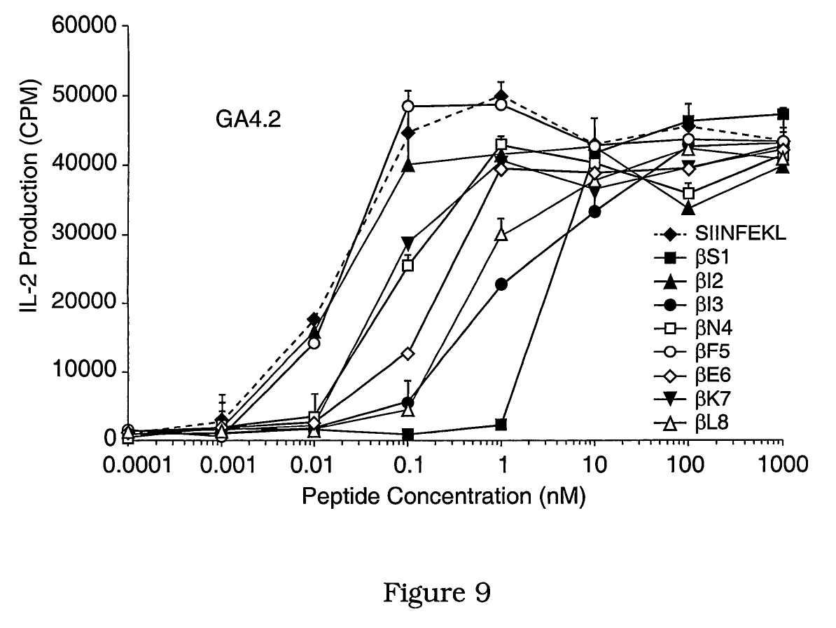

I-3 adherent antigen presenting cells (APC) were pre-plated ˜10⁵ cells per well overnight at 37° C. APC were pulsed with 1 nM of agonist SIINFEKL peptide (this concentration gave 50% maximal stimulation of the T hybridomas see results FIG. 9) and incubated for 1 hr at 37° C. Graded concentrations (10⁻¹³M to 10⁻⁶M) of β-analogues were then added and incubated for an additional 1 hr. T-hybrid clones grown to ˜10⁶ cells/ml were washed and resuspended at a density of 10⁵ cells per well, then transferred to wells containing the pulsed I-3 cells. T-hybrid clones and APC were then incubated in co-culture for 20-24 hrs. 50 μl of co-culture supernatant was harvested from each well and added to the CTLL-2 bioassay as in the T-hybrid assay. Antagonism was detected as a decrease in the basal level of IL-2 production at a concentration of agonist peptide resulting in half maximal stimulation of the T hybrids.

Serum Stability Assay

10 μL of an aqueous peptide solution containing 1 mg/ml peptide was added to 100 μl of 20% mouse serum (From adult male BL6 mice), and incubated at 37° C. for 0 min, 2 hrs and 12 hrs. At each time point samples were taken and serum proteins removed by precipitation through the addition of 40 μl of 15% TCA. Samples were stored at 4° C. for 30 min and centrifuged. Supernatants were then removed and kept on ice. 50 μl of each supernatant was analysed by RP-HPLC using 0.1% TFA in water (eluent A) and 0.09% TFA in 60% aqueous acetonitrile (eluent B) and the following gradient.

Gradient:

0% B for 5 min

Linear gradient to 60% B for 35 min.

The column was a Pharmacia μRPC octadecyl silica column of 3 μM nominal particle size and 300 Å pore size. The flow rate was 200 μl/min and UV detection was used at 214 nm, 254 nm and 280 nm. Values are given as area of peptide peak (214 nm). Peptide amounts at T=0 min were used as 100%. Peptide digests were fractionated and Electrospray ionisation (ESI) Ion Trap Mass spectrometry was used to characterise the fragmentation pattern of SIINFEKL and selected β-amino acid analogues.

### EXAMPLE 2

### Effects of β_{C3}-Amino Acide Incorporation on MHC Binding Affinity

The response to SIINFEKL by CTLs in C57/BL6 mice is predominantly restricted through the MHC Class I allele H-2K^{b} (Porgador, A., J. W. Yewdell, Y. Deng, J. R. Bennink, R. N. Germain. 1997. Localization, quantitation, and in situ detection of specific peptide-MHC class I complexes using a monoclonal antibody. Immunity 6:715). To characterise the effect that β_{C3}-amino acids have on K^{b} stabilisation, mutant peptides of the SIINFEKL peptide containing single β_{C3}-amino acid substitutions were synthesised (Table 4). The peptides were tested for binding to the surface K^{b} on the TAP mutant cell line RMA-S.

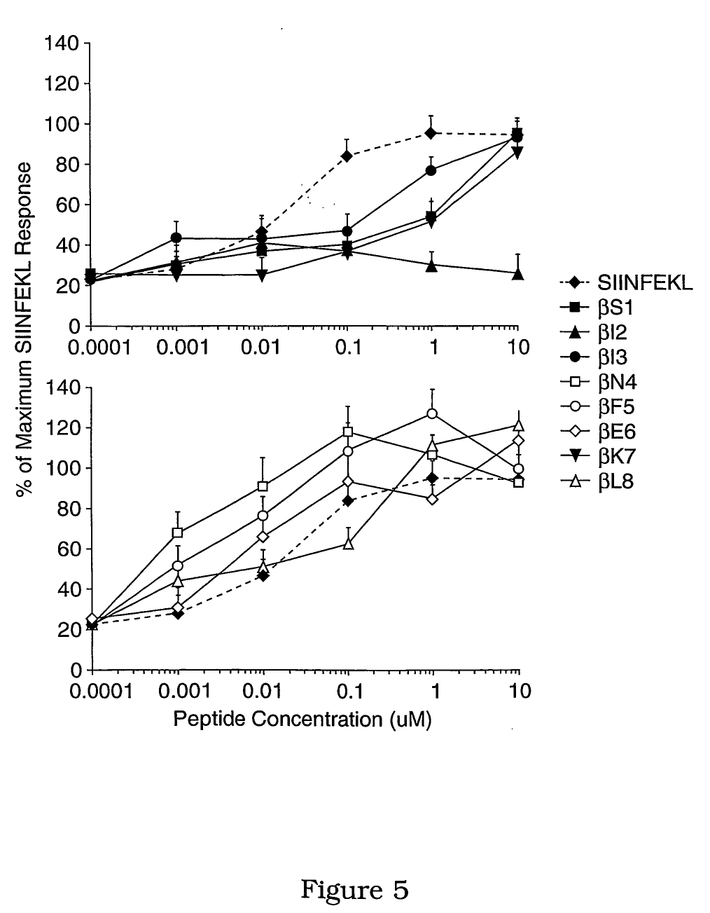

These cells express low levels of K^{b} (˜5%-10% of normal) at 37° C.¹⁹, but at incubation temperatures between 19° and 33° C. expression of surface K^{b} is amplified (˜50% of parental RMA cell line) (Ljunggren, H. G., N. J. Stam, J. J. C. Öhlen, P. Neefjes, M. T. Höglund, J. Heemels, T. N. Bastin, A. Schumacher, K. Kärre Townsend, H. L. Ploegh. 1990. Empty MHC class I molecules come out in the cold. Nature 346:476). These cold-induced molecules are quickly removed from the surface when the cells are returned to 37° C. unless stabilised by a K^{b} binding peptide. RMA-S cells were cultured overnight at 26° C. and tested for K^{b} expression, cells were then pulsed with serial dilutions of each peptide for an additional 1 hr before being placed at 37° C. for 2 hrs. Substitution at the N-terminus end of the peptide resulted in decreased binding affinity to the K^{b} molecule. However, substitution in the middle and C-terminus end resulted in equivalent or increased binding stabilisation of cold stabilised Kb molecules relative to the SIINFEKL peptide. Results were highly reproducible and representative data are shown in FIG. 5.

### EXAMPLE 3

### Effects of β_{C3}-Amino Acide Incorporation on Exposed Residues

The monoclonal antibody 25-D 1.16 specifically binds to the SIINFEKL peptide in conjunction with the K^{b} molecule. The SIINFEKL peptide is mostly buried within the MHC cleft with only the side chains of residues at P4, P6 and P7 facing outwards from the surface of the complex. This peptide binding orientation was first predicted by an alanine substitution scan (Jameson S. C., Bevan M. J., Dissection of major histocompatibility complex (MHC) and T cell receptor contact residues in a K^{b} -restricted ovalbumin peptide and an assessment of the predictive power of MHC-binding motifs. Eur J Immunol. October 1992; 22(10):2663-7) of SIINFEKL and then confirmed by the K^{b}/SIINFEKL crystal structure (Fremont D. H., Stura E. A., Matsumura M., Peterson P. A., and Wilson I. A. Crystal structure of an H-2Kb-ovalbumin peptide complex reveals the interplay of primary and secondary anchor positions in the major histocompatibility complex binding groove. Proc Natl Acad Sci USA. Mar. 28, 1995;92(7):2479-83). The residues Asn, Glu and Lys of SIINFEKL (P4, P6 and P7 respectively) are known to be involved in CTL recognition. To test the impact of incorporating β_{C3}-amino acids into SIINFEKL on the orientation of these exposed residues, the RMA-S stabilisation assay was repeated with the antibody 25-D1.16. Changes in 25-D1.16 recognition have previously been correlated with altered TCR recognition (Porgador et al, 1997, supra), suggesting this antibody has TCR like binding specificity.

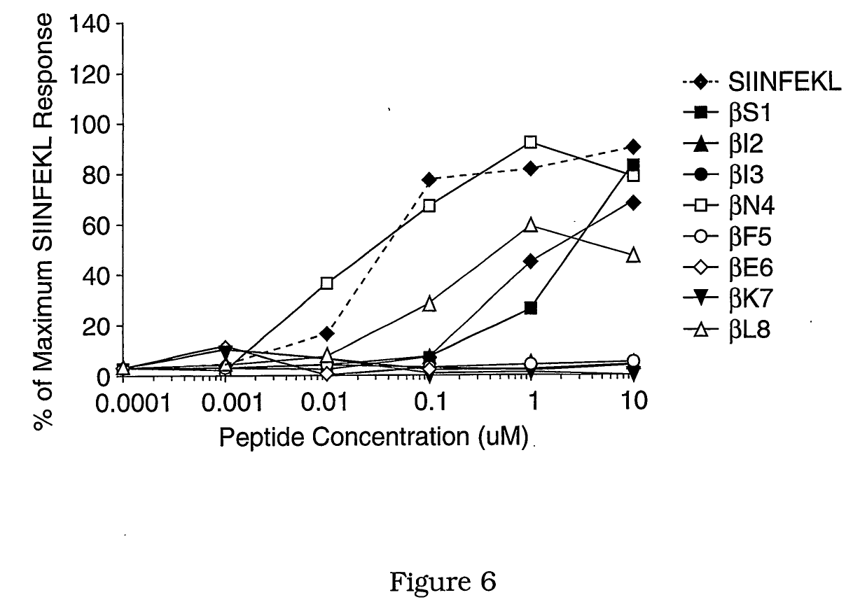

Substituting a β-amino acid into any part of the SIINFEKL peptide resulted in a change in the orientation or composition of solvent exposed residues. The most vulnerable positions for substitutions were P2 and P5, which abolished recognition by the 25-D1.16 antibody. Substitutions at P4 and P8 had the least effect, while peptides with substitutions at P1 and P3 needed a 100-fold increase in concentration to reach the same level of recognition as SIINFEKL. The results from the analysis were highly reproducible and representative data are shown in FIG. 6.

### EXAMPLE 4

### Effects of β_{C3}-Amino Acid Incorporation on CTL Recognition

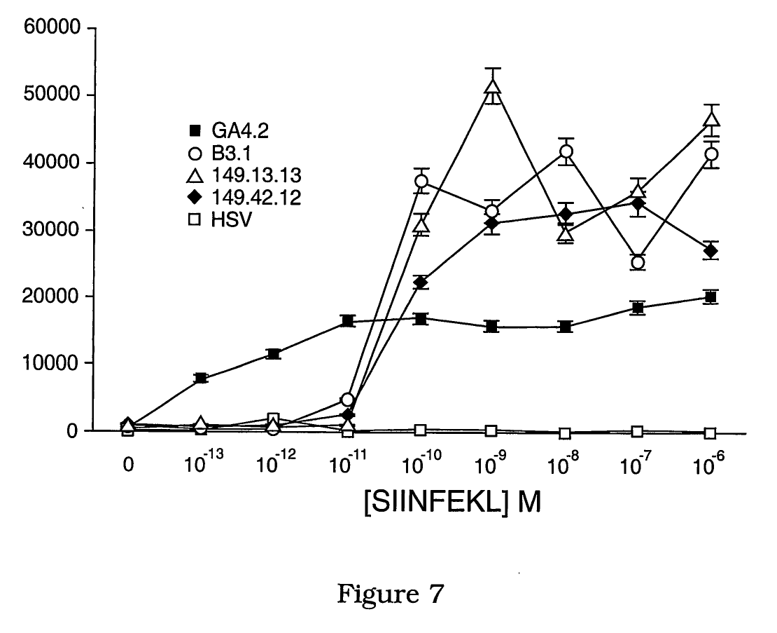

CTL clones (expressing different TCR Vβ/Vα segments) restricted by the H2-K^{b} molecule, and specific to the SIINFEKL peptide were tested for their ability to recognise the various β_{C3}-amino acid substituted analogues (Table X). A titration of the parental SIINFEKL peptide on each clone was completed to determine the optimal peptide concentration for CTL recognition (FIG. 7). I-3 cells were cultured overnight then pulsed with 1 μM of each of the β_{C3}-amino acid analogues for 1 hr. CTL clones were placed in co-culture with pulsed I-3 cells for 20-24 hrs. Supernatant from co-culture containing secreted IL-2 is then added to the IL-2dependent CTLL-2 cells. CTTL-2 proliferation is then measured by 3H-Thymydine incorporation and counted on a scintillation counter.

Differences were observed in the effect of substitutions at different positions. Analogues with substitutions at P1, P3, P4, P5 and P8 were least affected with considerable recognition from the CTL GA4.2, with approximately 50% recognition by B3.1 and 30% by 149.13.13. Although recognition by 149.42.12 was almost non-existent when compared to the parental peptide. The analogue with a P2 substitution resulted is significantly less recognition by all clones except GA4.2. At P6 (residue with side chain protecting group), no response was observed. The response seen in the control CTL clone HSV2.3 (Herpes Simplex Virus specific clone) is mainly mediated by the SSIEFARL peptide, as expected this peptide was not recognised by the K^{b}/SIINFEKL restricted clones. Concanavalin A was used to induce maximal IL-2 secretion in CTL clones as an indicator of CTL functionality.

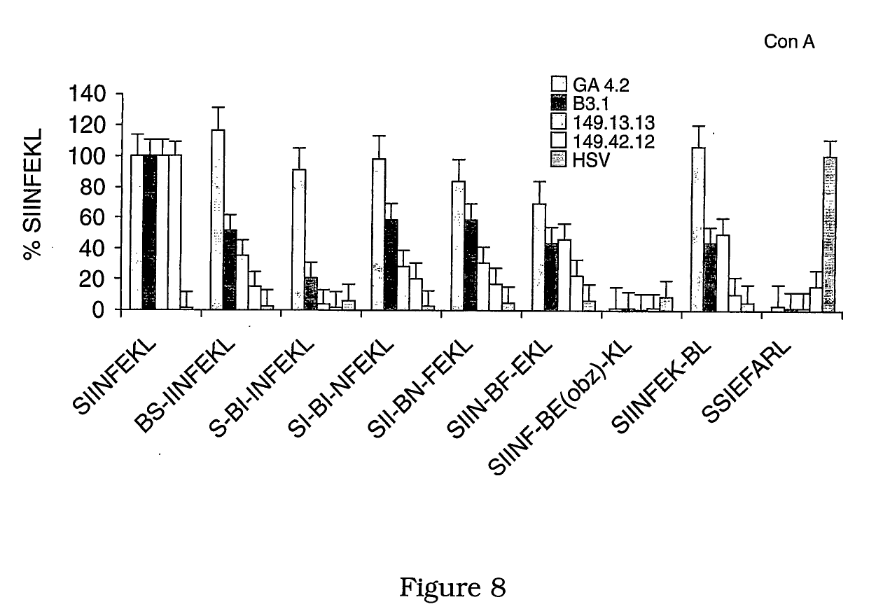

The effect of substituting β_{C3}-amino acids on CTL recognition had some correlation with stability of peptides to K^{b}. For instance β_{C3}-Ile at P2, which had the lowest affinity for K^{b} and also the least recognition by all CTL clones. Additionally, β_{C3}-Phe at P5, which had the strongest stabilising effect on K^{b} also had the most wide spread response to CTL clones. These results indicate that substitutions of β_{C3}-amino acids at individual positions of the SIINFEKL peptide generally decrease CTL recognition. This effect is dependent on the position of the substitution and does show a subtle correlation with stability of the K^{b}/peptide complex. Results were reproducible and representative data are shown in FIGS. 8 and 9.

### EXAMPLE 5

### TCR Antagonism of the K^{b}/SIINFEKL Specific GA4.2 and B3.1 CTL Clones

The antagonist assay was used to determine if the incorporation of β_{C3}-amino acids could alter the normal signalling and IL-2 secretion even in the presence of the agonist peptide. The peptide analogues were tested in an assay designed to distinguish TCR antagonism from competition with MHC binding. This involves prepulsing the target cells with a suboptimal dose of the SIINFEKL peptide before incubating them with the β-analogues and CTLs. The suboptimal concentration chosen of the natural SIINFEKL peptide was 1 nM which was based on the SIINFEKL titration assay (FIG. 7) where this concentration induced 50% of the maximal IL-2 production in these co-cultures. I-3 cells were pulsed with 1 nM of the parental SIINFEKL peptide. β-amino acid substitutions were then titrated in. CTL clones GA4.2 and B3.1 were then placed in co-culture with pulsed I-3 cells, and after incubation supernatant from this co-culture was removed and placed into an IL-2 bioassay as in the CTL recognition assay.

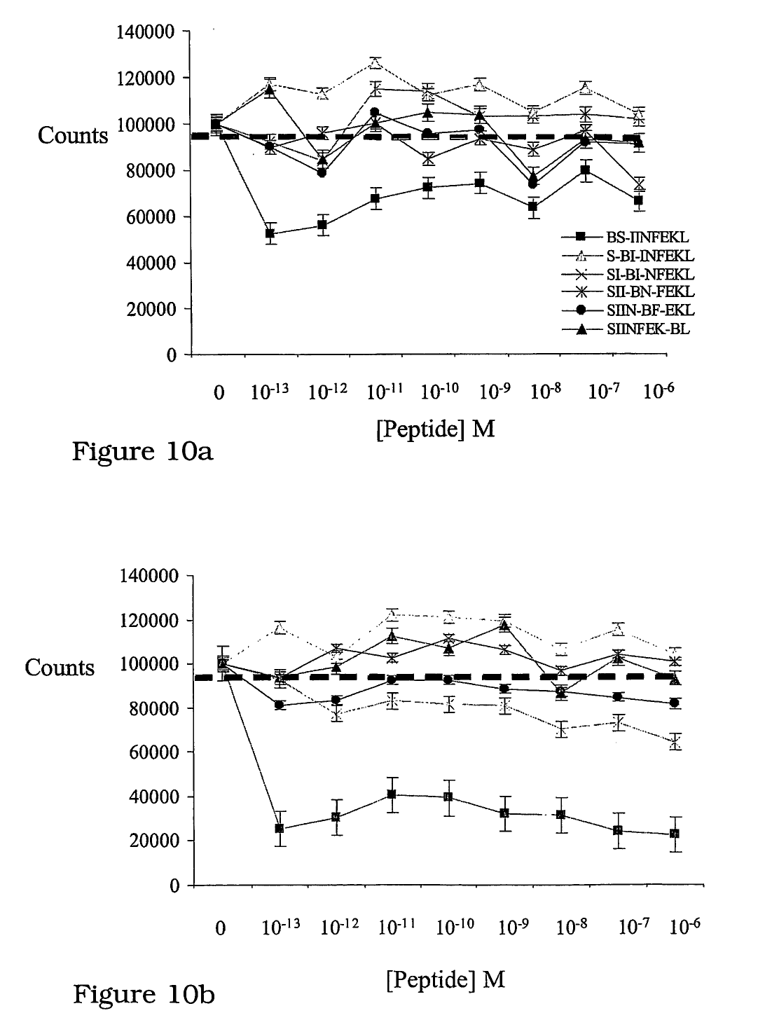

The incorporation of β_{C3}-amino acids into the SIINFEKL peptide resulted in two analogues that could modify the parental SIINFEKL response. Substituting β-Ile at P2 produced an analogue that increased the relative IL-2 secretion of both clones. In the previous CTL recognition assay the substitution of β-Ile at P2 resulted in decreased IL-2 secretion in both B3.1 and GA4.2 cells (FIGS. 8, 9), however when this peptide is co-administered with the parental SIINFEKL peptide it increased the IL-2 secretion above the SIINFEKL basal response (FIG. 10). Conversely, the second analogue, β-Ser at P1, was a strong antagonist of B3.1 and a weaker antagonist of GA4.2. In the previous CTL recognition assay β-Ser peptide analogues stimulated CTL clones with a relatively high efficacy (FIG. 8), but when co-administered with the parental SIINFEKL peptide it resulted in extensive antagonism of the parental SIINFEKL response. The fact that both these analogues altered the response in both cell lines demonstrates that the modifying TCR signalling by using β-analogues may not be a monoclonal event. This antagonism of the parental response was observed to occur down to 0.1 pM in both CTL clones. Results were reproducible and representative data are shown in FIG. 10.

### EXAMPLE 6

### Effects of β_{C3}-Amino Acid Incorporation on Mouse Serum Stability

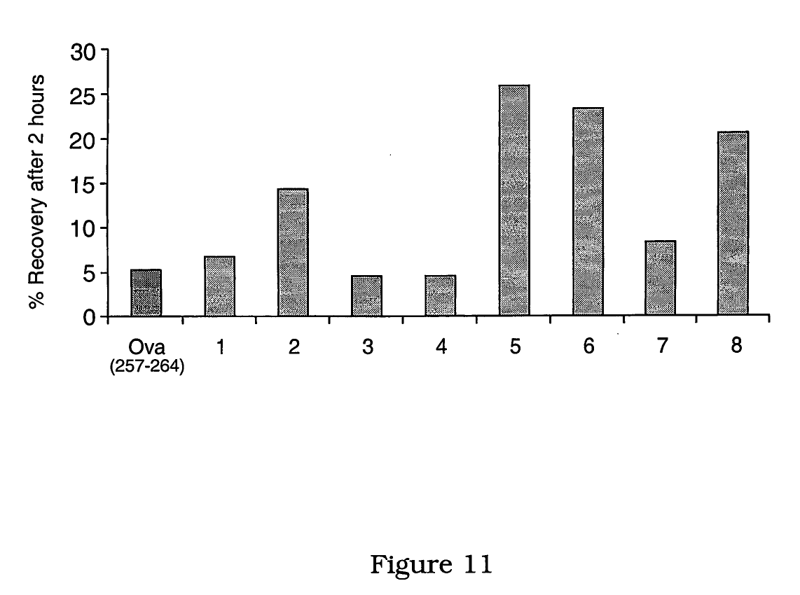

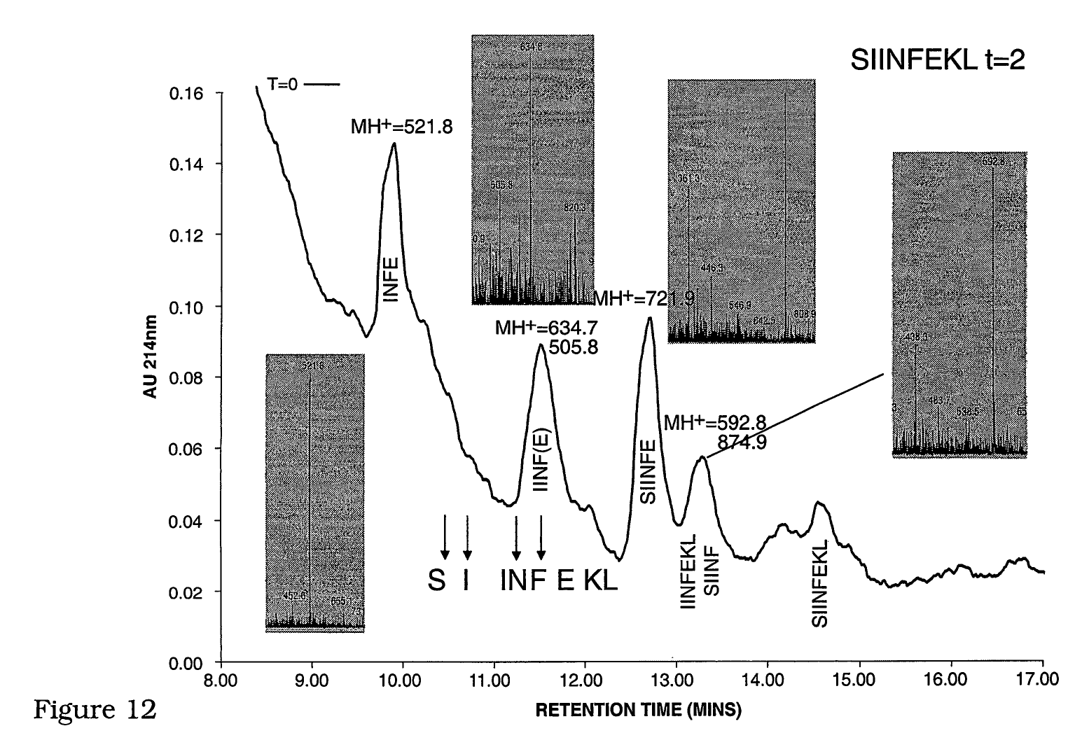

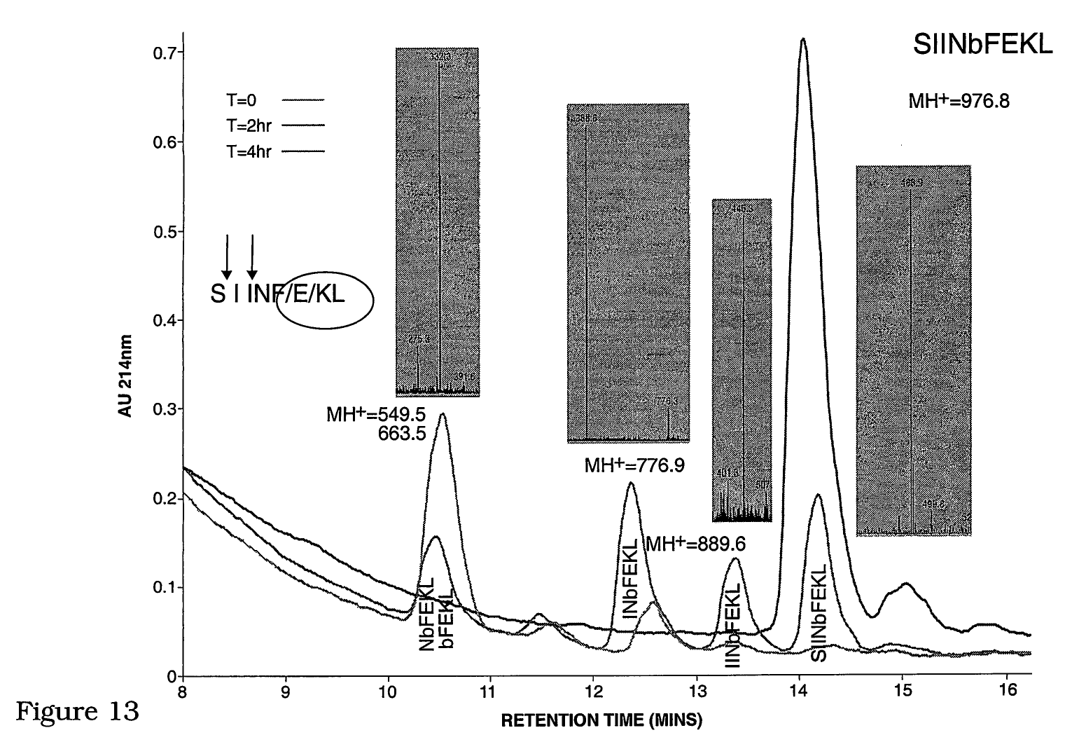

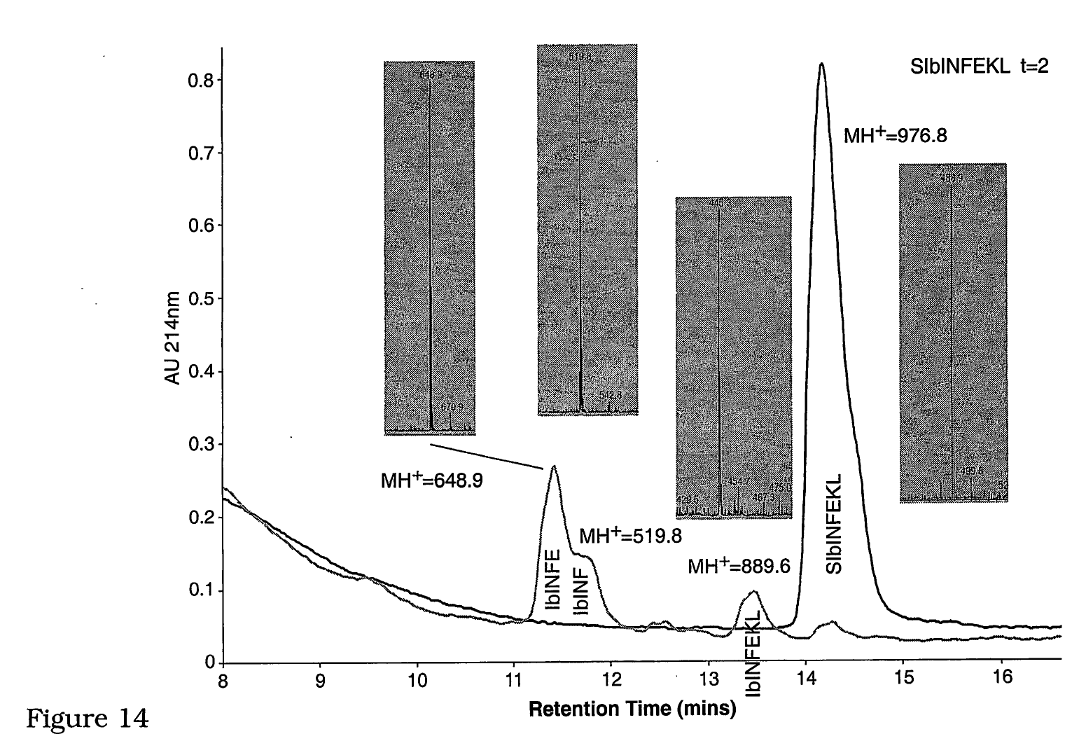

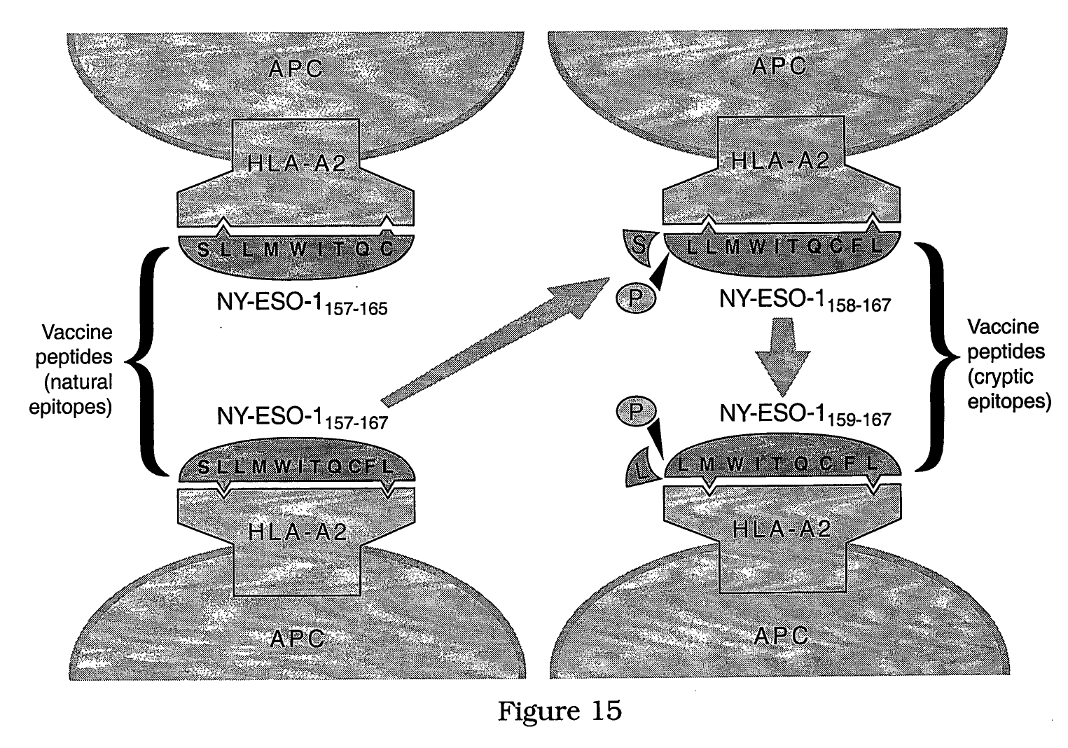

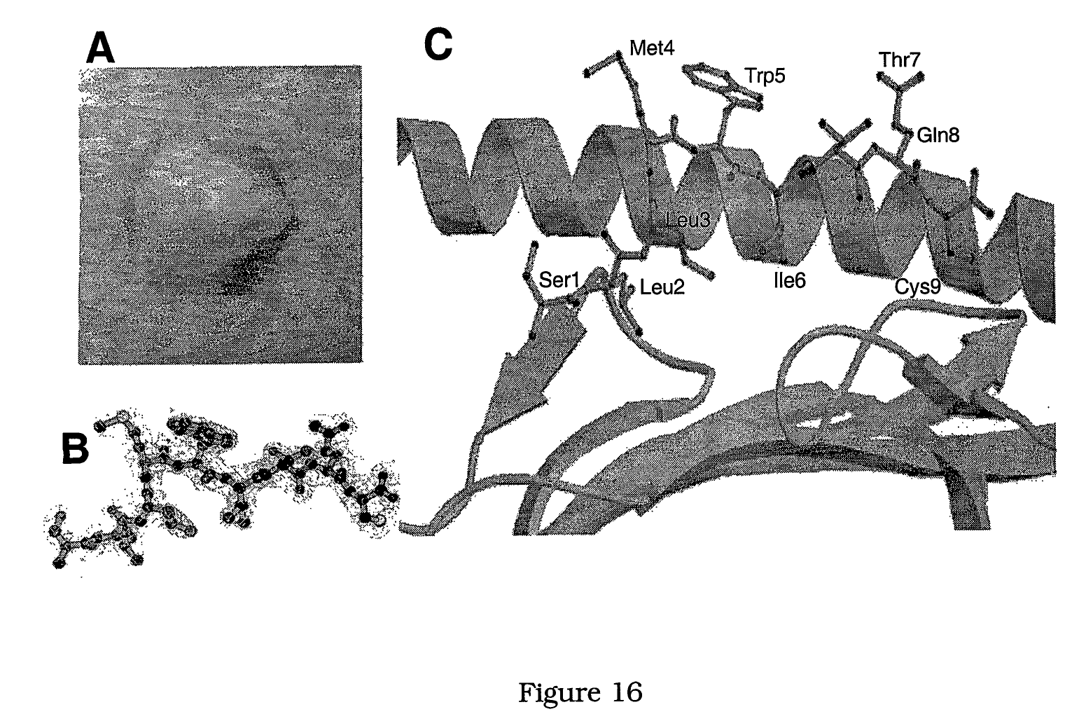

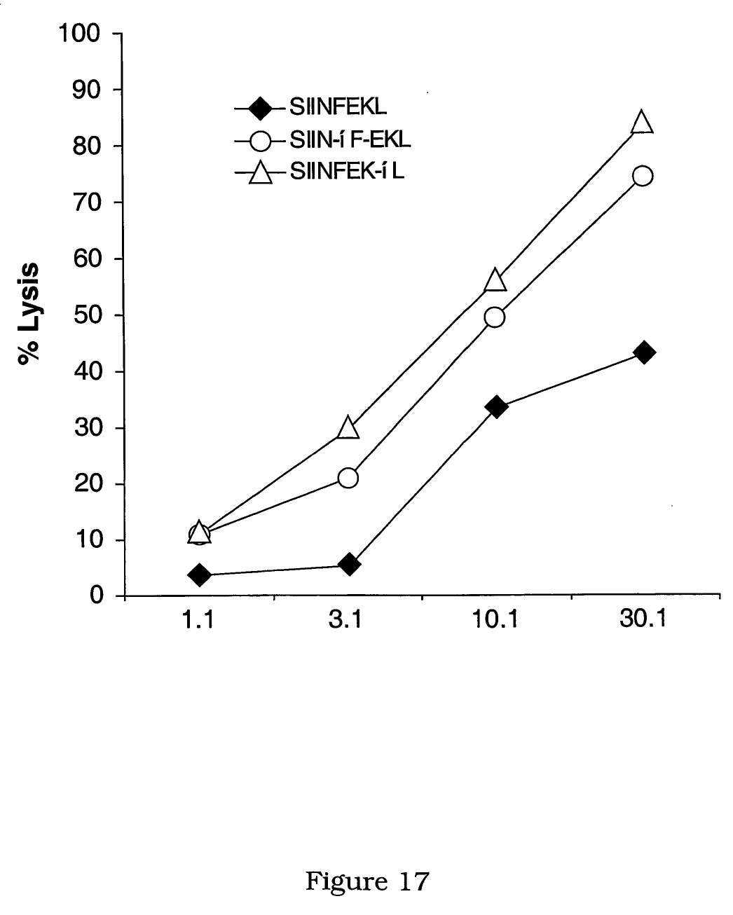

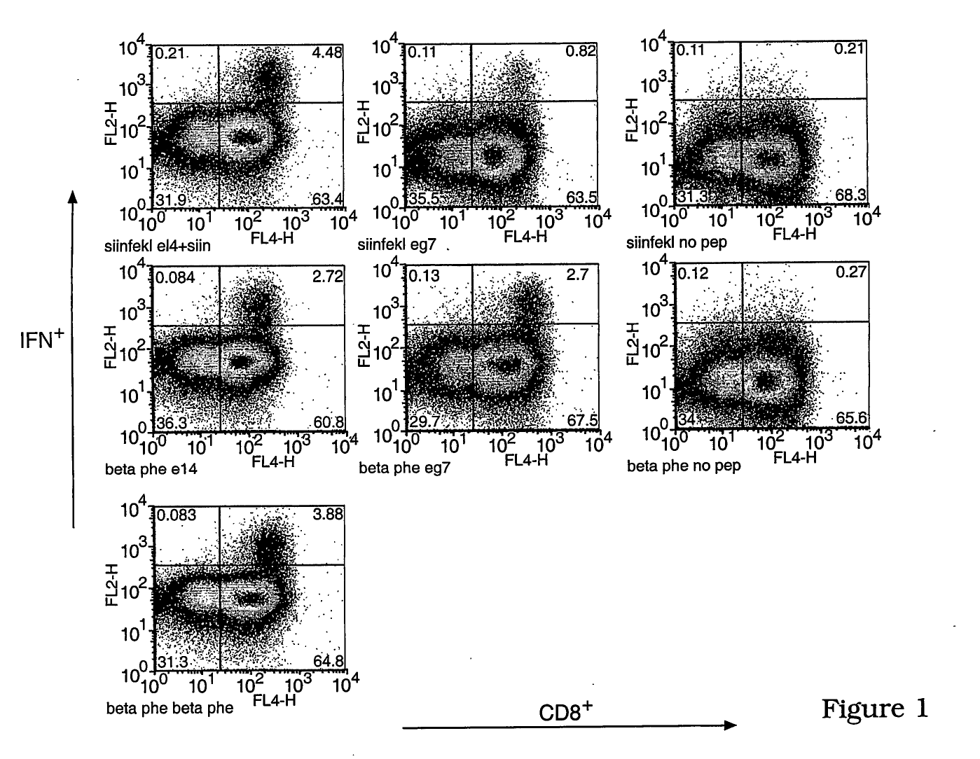

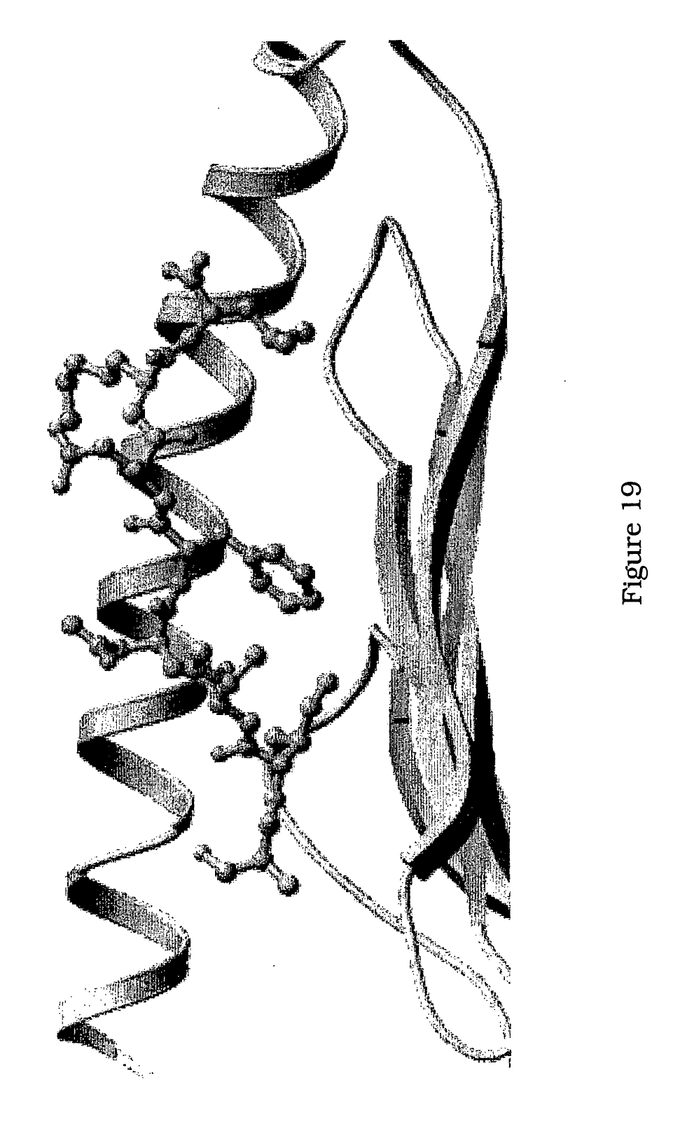

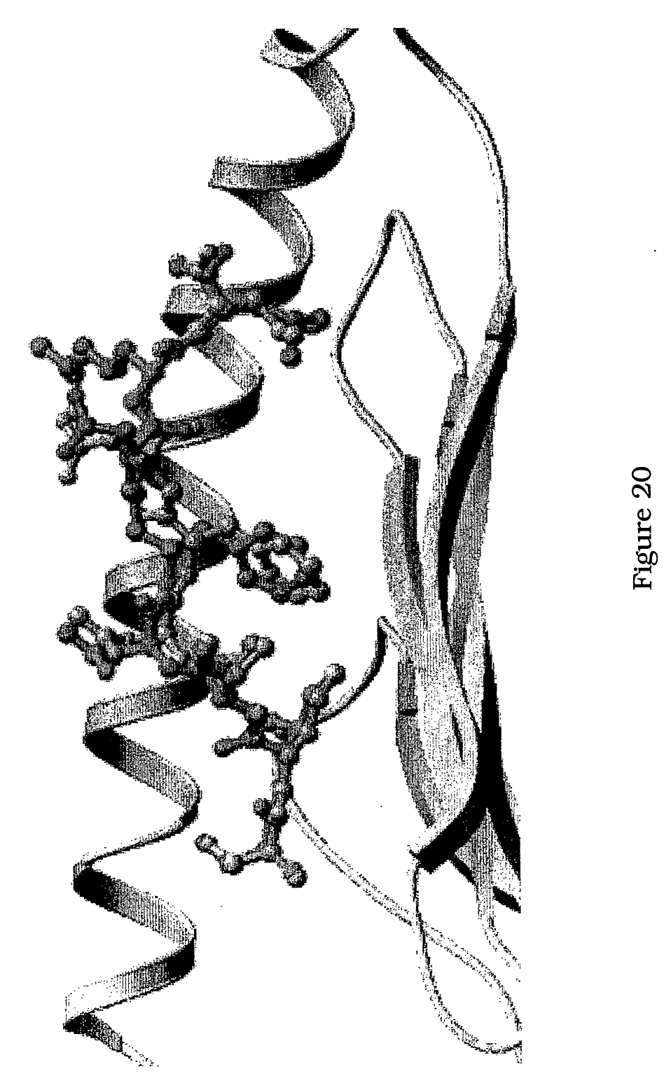

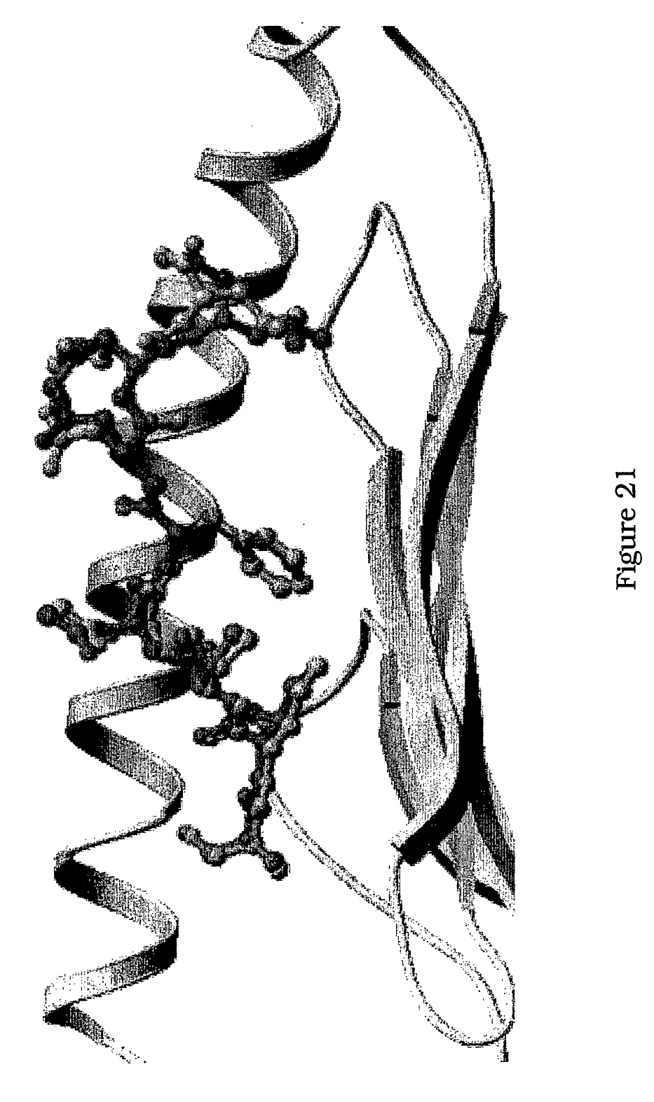

Since β-amino acids are resistant to proteolysis, the influence of peptide modification on stability against proteolysis was tested by monitoring the degradation of the peptides in mouse blood serum. Degradation kinetics were followed by reverse phase-HPLC (RP-HPLC) analysis using corresponding peak area for peptide quantification. The parental SIINFEKL peptide was degraded rapidly, with only 5% of the peptide being recovered after 2 hrs. For the β_{C3}-analogues, the amount of degradation was highly dependent on the position of the substitution. Limited stabilisation occurred for peptides with substitutions at P3 and P4 with 8% and 7% recovery respectively (FIG. 11). For the substitution at P1, 10% of the initial peak was recovered. However, a significant resistance to proteolytic degradation was formed with substitutions at P2, P5, and P8. For β-Phe at P5 and β-Leu at P8 over 25% of the initial peak was recovered after 2 hrs.

From the HPLC analysis it was observed that some fragments of degraded β-Ile (P3) and β-Phe (P5) peptides were accumulating over time (FIGS. 12-14). Mass Spectrometry and MS/MS were carried out on these peptide fragments as well as the parental SIINFEKL fragments. Together the results revealed the cleavage points from serum proteolysis, and also showed that the incorporation of the β-amino acid stopped cleavage at the position of substitution. Representative results are shown in FIGS. 12, 13 and 14.

### EXAMPLE 7

### NY-ESO-1

NY-ESO-1 a cancer testis antigen is expressed in many different types of tumors, including melanoma, breast, lung and bladder cancers but not in normal adult somatic tissue. In addition to its widespread expression by different cancers, it is also highly immunogenic eliciting both humoral and cellular immune responses in patients. Clinical evidence suggests that cytotoxic T lymphocyte specific for NY-ESO determinants can stabilize malignant disease and eradicate metastases, making NY-ESO an ideal tumor vaccine component. Peptide vaccination with NY-ESO determinants has been very promising, but along the way these studies have highlighted problems of stability and bioavailability associated with peptide immunization and the frequent failure to elicit robust CTL that kill tumors.

Response to NY-ESO in Cancer Patients and Vaccine Recipients

NY-ESO is highly immunogenic in cancer patients with advanced disease, where both cellular and humoral responses are evident. Both Class I and Class II restricted determinants have been identified making NY-ESO, or peptides derived from it, useful vaccine components. Moreover, the HLA-A2-restricted response to NY-ESO has been particularly well characterised and focuses on the 155-167 region of the antigen. Peptides from this region are currently used in clinical trials at the Ludwig Institute of Cancer Research (LICR). To date, vaccination with NY-ESO peptides has generally elicited poorly tumor reactive anti-peptide T_{CD8}. This appears to be associated with poor antigen availability, cleavage of the N-terminus of the peptide antigen to generate unnatural HLA-A2 ligands (i.e. cryptic epitopes) and potential oxidative degradation of the peptide due to the presence of Cys and Met residues. Three peptides from an overlapping region of the NY-ESO protein (155-163 QLSLLMWIT (SEQ ID NO: 9), 157-165 SLLMWITQC (SEQ ID NO: 10), and 157-167 SLLMWITQCFL (SEQ ID NO: 11)) have previously been reported as HLA-A*0201—restricted determinants recognized by tumor-reactive T_{CD8} from a melanoma patient. Despite poor binding to HLA-A2 tumor-reactive T_{CD8} clones mainly recognise the ESO₁₅₇₋₁₆₅ determinant. The immunogenicity of NY-ESO peptides was first evaluated in a trial vaccination of cancer patients in which a mixture of these peptides were administered intradermally to patients bearing NY-ESO⁺ tumors A vigorous CD8⁺ T cell response to ESO₁₅₇₋₁₆₇ was observed, whereas reactivity against ESO₁₅₇₋₁₆₅ appeared later and at a lower level. The T_{CD8} response to NY-ESO peptide vaccination has also been examined by HLA-A2/peptide tetramer analysis and revealed a heterogeneous response directed against several distinct overlapping epitopes, including cryptic determinants generated from aminopeptidase activity (FIG. 15). The cryptic ESO₁₅₉₋₁₆₇ determinant has also been and it does not appear to be naturally presented by tumor cells. Only cytotoxic T lymphocyte recognizing the precise ESO₁₅₇₋₁₆₅ determinant also recognize the endogenously processed determinant on NY-ESO⁺ tumor cells. Thus, it appears that only ESO₁₅₇₋₁₆₅ immunity produces high avidity anti-tumor cytotoxic T lymphocyte. The observation that this shorter peptide constitutes the clinically relevant epitope highlights its importance.

Previous Modification of NY-ESO Determinants Fail to Elicit Robust Tumor Reactive Cytotoxic T Lymphocyte

Analogs of ESO₁₅₇₋₁₆₅ where the C-terminal Cys residue has been replaced with more conventional anchor residues, namely I9, L9 and V9 analogs have been generated. Whilst these analogs bind more efficiently to HLA-A2 and are recognised by some cytotoxic T lymphocyte raised against the natural ESO₁₅₇₋₁₆₅ peptide, they do not induce high quality anti-tumor cytotoxic T lymphocyte in vivo. Indeed, the presence of the Cys at the C-terminus is critical for generating cytotoxic T lymphocyte that recognise endogenously processed NY-ESO determinants on tumor cells. The presence of this amino acid causes problems with formulation due to oxidative damage and dimerisation, both of which reduce the efficacy of the peptide antigen as an immunogen. The exact role of the Cys residue and in particular the reactive thiol can be visualised in the recently solved high resolution structure of this peptide complexed to HLA-A2 (see next section), which defines the role of the thiol in providing anchor interactions, its influence on antigen binding cleft conformation and potential TcR interactions. Moreover, the biochemical nature of the naturally processed and presented NY-ESO determinants remains obscure and has only been probed by the reactivity patterns of cross-reactive cytotoxic T lymphocyte. The identity of naturally processed NY-ESO determinants present on the surface of tumor cell lines and on professional APC is investigated.

As seen in NY-ESO peptide vaccination, proteolysis can effect the precision with which peptide immunogens can be delivered to antigen presenting cells leading to the presentation of cryptic epitopes that can divert the intended immune response towards more futile reactivities. Engineering protease resistance and stability into peptide based epitopes in conjunction with suitable delivery vehicles represents the way forward in vaccine design, allowing the precise presentation of antigenic determinants by APC. An integrated approach that combines peptide and organic chemistry, structural biology and studies of the ensuing immune response enables the rational design of epitope based immunotherapeutics. Precise delivery of antigenic determinants is a property of relevance to vaccination intended to elicit anti-tumor and anti-viral immunity as well as vaccination to ameliorate autoimmunity or block transplant rejection.

Experimental Design

(1) The presentation of peptide determinants derived from NY-ESO is examined and new HLA-restricted determinants of relevance to anti-tumor immunity are identified.
(2) The high resolution structures of HLA-A2-NY-ESO peptide complexes of direct relevance to vaccination studies are determined and their structures correlated with their ability to induce anti-tumor activity.
(3) The structures of anti-tumor TcR complexed to NY-ESO peptide-HLA-A2 are examined to define important elements of the recognition process that elicits anti-tumor immunity.
(4) New peptidomimetics based on the tumor antigen NY-ESO with improved protease resistance and stability, based on the current invention, that allow precise presentation of antigenic determinants are designed.
(5) Immunogenicity studies of the NY-ESO based peptidomimetics using cell lines from cancer patients and vaccine recipients as well as HLA transgenic mice are performed.

These studies identify the biochemical nature of naturally processed NY-ESO determinants restricted by multiple alleles and examine the processing requirements of exogenous peptide antigen.

The generation of protease resistant and stable NY-ESO analogs has high clinical significance. Of importance to these designs is the generation of high resolution structures of HLA-ESO peptide complexes (which is now routine procedure), as they form the structural template for analog design.

High Resolution Structures of NY-ESO Epitopes Restricted by HLA-A2

The structure of HLA-A2 complexed to ESO₁₅₇₋₁₆₅ (SLLMWITQC) to 2.1 Å resolution has been solved (FIG. 16). The electron density for the peptide ligand is unambiguous and clearly shows the solvent accessibility of Met-4, Trp-5, Thr-7 and Gln-8, indicating that these residues would play a key role in TcR recognition. The Cys-8 residue is buried and participates in anchoring interactions with the hydrophobic F pocket. This indicates that the thiol is not critical for binding, however, comparison of this structure to A9, V9 or I/L9 analogs should reveals the role the thiol plays in modulating the antigen binding cleft conformation.

Identification of NY-ESO Determinants Presented by Tumor Cells and Professional APC

The normal endogenous array of peptide determinants processed and presented from NY-ESO on tumor cells and on professional APC is investigated. Following vaccination NY-ESO determinants that are naturally presented by tumor cells (spanning residues 157-167, 155-163, 157-165) and cryptic determinants that are only presented as a result of peptide vaccination (158-167 and 159-167) elicit anti-peptide cytotoxic T lymphocyte. Only those peptides naturally presented by tumor cells and in particular the ESO₁₅₇₋₁₆₅ determinant are clinically useful.

(i) Isolation of Endogenously Processed HLA-A2 Bound Peptide Ligands from NY-ESO

The process of isolating MHC Class I and Class II bound peptide ligands involves large scale cell culture (5×10⁹-10¹⁰ cells), isolation of detergent solubilized MHC complexes using immobilized specific mAb, elution of complexes in acid, and ultrafiltration to remove MHC proteins from the dissociated peptide ligands. The isolated peptides are separated by multi-dimensional reversed phase HPLC and the fractions analysed for biological activity (e.g. cytotoxic T lymphocyte assays) and individual peptides identified by mass spectrometry. Peptide elution from HLA-typed human NY-ESO⁺ tumour cell lines uses well-established procedures. Known HLA-A2-resricted epitopes which can be identified by mass (or MS/MS fragmentation) and chromatographic retention time are searched for their natural presentation in tumor cells and APC are confirmed. In addition, the identity of these fractions is screened functionally using cytotoxic T lymphocyte lines, tumor infiltrating lymphocytes (TIL) or PBMC from patients using routine cytolysis and cytoline based assays. HLA-A2-complexes are purified using the specific mAb BB7-2, the A2-depeleted material is then subjected to further rounds of purification to enrich for other class I (e.g. e have mAbs specific for the Bw4 and Bw6 determinants) or class II alleles (e.g. mAbs L243 (DR), SPVL3 (DQ) or B.721 (DP)). Eluates from these alleles are examined as a second priority to the HLA-A2-derived material but should yield new and clinically useful determinants.

(ii) Processing of NY-ESO Determinants by Dendrific Cells

This proceeds along similar lines following antigen or peptide feeding of dendritic cells prepared either from pooled individual samples (10-20 buffy coat preparations will yield approximately 10⁹ dendritic cells) or bone marrow dendritic cells prepared from the HLA-A2 transgenic mice. The dendritic cells are pulsed with various NY-ESO peptides or recombinant ESO. Preliminary experiments using recombinant ESO has demonstrated that 20 μg/ml loads 2-5×10⁶ dendritic cells allows stimulation of specific T cell cultures. This is scaled up to provide sufficient material for peptide elution. A construct that expresses an ER-targeted ESO₁₅₇₋₁₆₇ generated ESO₁₅₇₋₁₆₅ and ESO₁₅₉₋₁₆₇ as determined by elution and T cell screening of RP-HPLC fractions. These fractions are examined by LC-MS/MS to confirm the identity of these peptides. Of particular interest is the examination of Cys-9 or Met-4 modifications since these are potential oxidative modifications of the peptide and treatment of the peptide/antigen presenting cell with reducing agents prior to in vitro assays has shown up to 10-fold improvements in cytotoxic T lymphocyte recognition. In addition, the conditions under which peptide loading of dendritic cells result in presentation of cryptic NY-ESO determinants (i.e. ESO₁₅₈₋₁₆₇ and ESO₁₅₉₋₁₆₇). Analogs of these peptides are examined for their ability to generate the clinically relevant ESO₁₅₇₋₁₆₅ and ESO₁₅₇₋₁₆₇ determinants using this approach.

High Resolution Structures of NY-ESO Epitopes Restricted by HLA-A2 and the Molecular Basis of T Cell Recognition of Tumour Cells

The high resolution structures of HLA-A2 with NY-ESO peptides (including, 157-167, 159-167 and 155-163) as well as previously published high affinity analogues in which the C-terminal Cys residue and of ESO₁₅₇₋₁₆₅ has been mutated to the more appropriate A2 anchor residue Ile or Val are solved. The role of this Cys residue in anchoring the peptide, the change in register of peptide binding in the cleft of the longer and cryptic peptides and their influence on cleft conformation is important for the design of analogs. Co-complexes of HLA-A2-ESO₁₅₇₋₁₆₅ and other naturally processed determinants with anti-tumour TcR are examined. These studies confirm which amino acid side chains make up important tumour recognition elements and allow fine tuning of mimic design.

Structure Guided Design of Peptidomimetics Based on Naturally Presented NY-ESO Determinants

A similar β-amino acid scan, to that shown for the SIINFEKL epitope is performed for ESO₁₅₇₋₁₆₅ and ESO₁₅₇₋₁₆₇. The effects of β-amino acid substitution on proteolytic stability, generation of immunogenic determinants and anti-tumor cytotoxic T lymphocyte-cross-reactivity are examined. Presentation of ESO₁₅₇₋₁₆₅ without liberation of cryptic epitopes associated with N-terminal trimming is obtained. Other epitopes may be studied as they come on line. Modification of the oxidatively sensitive Met and Cys residues is examined. C-terminally modified analogues are designed by replacing the Cys residue with the isosteric L-Amino butyric acid (Abu) or Ser. The structure allows the rational replacement of this residue to ensure the fidelity of the MHC-peptide complex and the elements important for TcR recognition of tumor cells are maintained.

Immunogenicity Studies of the Peptidomimetics

The evaluation of the immunogenicity of NY-ESO analogs is performed initially using human cytotoxic T lymphocyte clones and lines, tumor infiltrating lymphocytes or PBMCs from patients with NY-ESO⁺ tumors. The criteria are cross-recognition of tumor cells and the analog pulsed onto APC (e.g. T2, autologous PBMCs). Promising analogs are tested for immunogenicity using HHD (HLA-A2.1 transgenic on a H-2 class I knockout background) mice in immunisation studies, since these mice represent the best pre-clinical model of human immunity. HHD mice are immunized with NY-ESO and analog peptides and their efficiency to generate cytotoxic T lymphocyte responses is monitored by cytotoxicity, ELISPOT assays and intracellular IFN-γ assay. Equally, peptide specific HLA-tetramers are used for the evaluation of the CD8⁺ specific lymphocytes generated from these cultures and for the phenotypic characterization of their activation status. T cells elicited towards the peptide analogs are tested for their ability to lyse NY-ESO positive tumor cell lines. Both the method of delivery and composition of the immunogen influences the avidity of anti-peptide cytotoxic T lymphocyte and ultimately their anti-tumor reactivity. Moreover, to elicit protective anti-tumor immunity it is sometimes necessary to use multiple determinants from NY-ESO and other tumour antigens.

### EXAMPLE 8

### Methods and Techniques

Preparation of Soluble Class I HLA-Peptide Complexes

Soluble class I heterodimers containing a specific peptide ligand are prepared by expressing truncated forms (amino acid residues 1-276) of the MHC heavy chain (hc) and full length β2-microglobulin (β2 m) in E. coli and each protein purified from inclusion bodies as we have described in detail elsewhere^{AP19}. The refolded complexes are purified by gel filtration chromatography and anion exchange chromatography to a high level of purity suitable for crystallographic studies.

Crystallization

The hanging drop method of protein crystallization is generally employed, although other methods such as sitting drop and dialysis buttons are available if necessary. In the hanging drop method, multi-well tissue culture plates are covered with a coverslip onto which a drop (containing 2-10 μl of protein solution mixed with an equal volume of reservoir solution) is deposited suspended over 0.7 ml of precipitant solution. Most HLA alleles have been crystallised using polyethylene glycol as a precipitant and published conditions will be used as starting points for these screens. Once crystals are obtained, fine-tuning of the initial crystallization conditions, micro- and macro-seeding are methods used to increase the size and quality of the crystal.

Isolation of TcR cDNAs and Construction of Expression Plasmids

RNA is prepared from A2-NY-ESO-restricted anti-tumour cytotoxic T lymphocyte with Trizol (Life Technologies), and reverse transcribed. DNA encoding either the TcR α or β chain is obtained by PCR amplification of cDNA using combinations of specific 5′ and 3′ primers and cloned into the pET-30 expression vector (Novagen). The predicted translation product from each DNA fragment lacks the leader sequence and translation is terminated immediately before each of the α or β chain constant region cysteines that normally forming an interchain disulphide bond. The codon encoding the unpaired cysteine at position 186 of the C β region is changed to encode alanine by site-directed mutagenesis.

X-Ray Crystallography

This is performed at the Protein Crystallography Unit, Monash University, Department of Biochemistry and Molecular Biology. All in-house X-ray measurements are made using a Rikagu RU-3HBR rotating anode generator with helium purged OSMIC focusing mirrors as an X-ray source. Data are collected using an R-AXIS IV++ detector. Crystals are routinely flash frozen (to a temperature of 100K) prior to data collection using the inverse phi geometry with an Oxford cryosystem in order to reduce the effects of X-ray induced radiation damage. In addition, the laboratory frequently makes visits to synchrotron facilities in the United States, where more intense X-rays are available. The three-dimensional diffraction data is processed and analysed using the HKL program suite and scaled using SCALEPACK. In addition there is a suite of Graphics workstations to undertake the stages of refinement, model building and analysis.

Structure Determination and Refinement

The structures are solved by molecular replacement and model building are performed using the tools within the software package ‘O’. Iterative rounds of model building and refinement are required to yield a final model. Refinement will proceed using CNS or programs within the CCP4 suite. The progress of refinement is monitored by the Free R value. As a general rule, bulk solvent corrections will be applied when refining the models. The choice of B-factor refinement (overall, grouped B-factors or individual B-factor) depends on the resolution of the structure. The validity of the structures are tested by such programs as PROCHECK, OOPS, 3D-PROFILE and WHATCHECK (reviewed in Dodson et al., 1998). There are a multitude of software packages available (MOLSCRIPT, RIBBONS, Midas Plus, PROMOTIF, programs within CCP4) to display and analyze the structure.

Evaluation of HLA Binding

HLA binding is examined using epitope stabilisation assays based around the stabilisation of conformational determinants on TAP-deficient APC such as T2 and transfectants as well as using micro-assembly assays of recombinant HLA, β2-microglobulin and peptide using either mass spectrometry of eluted peptides or capture ELISA with a conformationally sensitive mAb as a readout.

Evaluation of CD8 Epitopes in vitro and in vivo

For in vitro studies human cell lines are used from patients vaccinated with NY-ESO peptides and those clones previously published by colleagues at various branches of LICR, which are tested for their recognition of the various analogs generated in this study using 51 Cr-release cytolysis experiments and intracellular IFN-γ staining experiments.

For in vivo studies HHD (HLA-A2.1 transgenic on a H-2 class I knockout background) mice (available at LICR) are used in immunisation studies since these mice represent the best pre-clinical model of human immunity. HHD mice are immunized with NY-ESO and analog peptides (formulated initially in IFA with the addition of an appropriate T helper epitope) and their efficiency to generate cytotoxic T lymphocyte responses is monitored by cytotoxicity, ELISPOT assays and intracellular IFN-γ assay. Equally, peptide specific HLA-tetramers are used for the evaluation of the CD8⁺ specific lymphocytes generated from these cultures and for the phenotypic characterization of their activation status. T cells elicited towards the peptide analogs are tested for their ability to lyse NY-ESO positive tumor cell lines in cytolysis assays.

### EXAMPLE 9

Chromium Release Assay

Results are presented of a standard chromium release assay (FIG. 17) and an intracellular cytokine staining assay (FIG. 18).

Effector cells were obtained from C57BL/6 mice immunised with 100 μg SIINFEKL, SIINβFEKL or SIINFEKβL in complete Freunds adjuvant 7 days previously. Spleen cells were depleted of erythrocytes by treatment with tris-buffered ammonium chloride and antigen specific T cells were restimulated in vitro for 13 days using irradiated SIINFEKL pulsed splenocytes from naive C57BL/6 mice.

Target cells used in CTL assays were EL4 (H-2b) thymoma cells or EG-7 (EL4 transfected with the Ovalbumin antigen facilitating physiological presentation of the native SIINFEKL determinant). Cells were labelled by resuspending in 200 μL 5% FCS v/v in RPMI containing 200 μCi 51 Cr (Amersham) and incubated for 2 hours at 37° C. After washing cells 3 times, concentration was adjusted to 10⁵ cells/mL with RPMI and cells dispensed in 100 μL aliquots into 96-well U-bottom TC plates. Serial dilutions of 100 μL aliquots of effector cells were added in triplicate. After four hours incubation at 37° C./5%CO2, 25 μL of the supernatant of each well was harvested onto Luma plates (Packard) and assayed for ⁵¹Cr by a γ-counter (Packard Topcount NXT). The specific ⁵¹Cr release at each effector: target ratio was calculated by subtraction of the cpm released spontaneously in wells containing target cells incubated with medium only. These values were then expressed as a percentage of cpm representing ⁵¹Cr released in samples from wells in which the target cells were incubated in 1% Triton X-100 (Total releasable cpm). Spontaneous release ranged from 1 to 10% of total releasable cpm. Data are presented as the mean of the values obtained from triplicate cultures.

Peptide Structure

Recombinant H-2K^{b} molecules were expressed in E. coli as inclusion bodies as described [Garboczi, D. N., Madden, D. R. & Wiley, D. C. Five viral peptide-HLA-A2 co-crystals. Simultaneous space group determination and X-ray data collection. J Mol Biol 239, 581-7 (1994)] using the BL21 (RIL) strain of Escherichia coli. The class I heavy chain was modified by the removal of the leader sequence, transmembrane region and cytosolic tail (amino acids 1-276 of the mature protein sequence). cDNA encoding this region was ligated into the bacterial expression vector pET, and transformed into the BL21 (RIL) strain of Escherichia coli. At an A600 of 0.6, cultures were induced with 1 mM of isopropyl-1-thio-β-D-galactopyranoside (IPTG) for 12 hours, bacteria were lysed in 50 mM Tris-HCl pH 8.0, 1% TritonX-100, 1% Sodium deoxycholate, 100 mM NaCl and 10 mM DTT. Inclusion bodies were isolated by centrifugation after washing with 50 mM Tris-HCl, 0.5% TritonX-100, 100 mM NaCl, 1 mM NAEDTA, 1 mM DTT, pH 8.0, and washing in 50 mM Tris-HCl, 1 mM NaEDTA, 1 mM DTT, pH 8.0, and then solubilized in 25 mM MES, 8M Urea, 10 mM NaEDTA, pH 6.0 with the protease inhibitors 1 μg/ml Pepstatin A and 200 μM phenylmethylsulfonyl fluoride (PMSF). Recombinant protein (60 mg Kb heavy chain and 20 mg β2 m) was refolded with 30 mg of the peptides (SIINβFEKL and SIINFEKβL) in the presence of 3M guanidine-HCl, 10 mM NaAcetate, and 10 mM NaEDTA, pH 4.2, over 24 hours in 0.1 M Tris, 2 mM EDTA, 400 mM L-Argilline-HCl, 0.5 mM Oxidized Glutathione, 5 mM Reduced Glutathione pH 8.0 at 4° C. Following refolding, protein was dialyzed overnight against Milli Q using a 6-8,000 kDa MWCO dialysis membrane (Spectrum, Calif., USA). Protein was concentrated by ion exchange on a DE52 column (Whatman, Maidstone, Kent, U.K.), and subsequently purified by size exclusion on a Superdex 75 pg gel filtration column (Amersham Pharmacia Biotech, Uppsala, Sweden), and a final ion exchange on a MonoQ HR 5/5 column (Amersham Pharmacia Biotech). Quantitative analysis was based on comparisons to BSA protein standards using SDS-polyacrylamide gel electrophoresis. Protein was concentrated to 3 mg/ml for use in crystallization trials.

All crystallization trials were conducted using the hanging drop vapour diffusion technique using 24-well tissue culture plates. The crystals were grown by mixing equal volumes of 3 mg/ml Kb-peptide complex with the reservoir buffer (0.1 M sodium cacodylate, 0.2 M calcium acetate, 14% (w/v) PEG 4000)) and microseeded from crystals grown in 16% (w/v) PEG 4000 conditions at room temperature. Each well contained 1 ml of reservoir buffer. The crystals belong to space group P21 with unit cell dimensions α=66.31 Å, b=89.46 Å, c=89.26 Å, β=111.46°. A 2.0 Å data set were collected using inverse phi geometry, processed and scaled using the HKL suite of programs. The structures were refined and an unambiguous molecular replacement solution was obtained. The electron density for the bound beta peptides was very clear (FIGS. 19-21).

Intracellular Cytokine Staining

1. APC were plated out at required density in a final volume of 100 μl/well
2. Effector cells were as for Chromium release
3. T cells were resuspended in RF-10 medium in round-bottom 96-well plate, 100 ml per well (2×10⁵/well) p0 4. Brefeldin A was added into each well to a final 5˜10 mg/ml
5. Incubated for further 3˜4 hours to let gIFN/IL-2 accumulate in ER in activated specific T cells
6. All cells harvested and stained with anti-CD8a on ice for 30˜60 min
7. Cells fixed with 1% paraformaldehyde in PBS, R.T for ˜20 min
8. Cells washed with 0.1% Saponin solution
9. Incubation in dark for 15 minutes
10. Cells pelleted by centrifugation and resuspended in 0.5% Saponin wash
11. Intracellular staining for gIFN or IL-2 at 1:100 dilution in PBS containing 0.1˜0.5% Saponin (final concentration, Calbiochem, #558255) on ice for 60 minutes
12. FACS analysis performed for two color with gating on the CD8+ cells among intact cell population

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Chang, H. C., A. Smolyar, R. Spoerl, T. Witte, Y. Yao, E. C. Goyarts, S. G. Nathenson, E. L. Reinherz. 1997. Topology of T cell receptor-peptide/class I MHC interaction defined by charge reversal complementation and functional analysis. J. Mol. Biol. 271:278.
Clark S. R., Barnden M., Kurts C., Carbone F. R., Miller J. F., Heath J. R. Characterization of the ovalbumin-specific TCR transgenic line OT-I: MHC elements for positive and negative selection. Immunol Cell Biol. April 2000;78(2):110-7.
Fremont D. H., Stura E. A., Matsumura M., Peterson P. A., and Wilson I. A. Crystal structure of an H-2Kb-ovalbumin peptide complex reveals the interplay of primary and secondary anchor positions in the major histocompatibility complex binding groove. Proc Natl Acad Sci USA. Mar. 28. 1995;92(7):2479-83.
Gillis, S., M. M. Ferm, W. Ou, K. A. Smith. 1978. T cell growth factor: parameters of production and a quantitative microassay for activity. J, Immunol. 120:2027.
Green, T. W. & Wutz, P., Protective Groups in Organic Synthesis, John Wiley & Son, 3^{rd} Edition, 1999.
Jameson S. C., Bevan M. J., Dissection of major histocompatibility complex (MHC) and T cell receptor contact residues in a K^{b}-restricted ovalbumin peptide and an assessment of the predictive power of MHC-binding motifs. Eur J Immunol. October 1992;22(10):2663-7.
Jones, B., Jr C. A. Janeway. 1981. Cooperative interaction of B lymphocytes with antigen-specific helper T lymphocytes is MHC restricted. Nature 292:547.
Jones, J., Amino Acid and Peptide Synthesis, Oxford Science Publications, 1992.
Karre K., Ljunggren H. G., Piontek G., and Kiessling R. Selective rejection of H-2-deficient lymphoma variants suggests alternative immune defence strategy. Nature. Feb. 20-26, 1986;319(6055):675-8.
Larock, R. C., Comprehensive Organic Transformations, 1989, VCH publishers.
Ljunggren, H. G. and Karre, K., (1985). Host resistance directed selectively against H-2-deficient lymphoma variants. Analysis of the mechanism. J Exp Med. December 1;162(6):1745-59.
Ljunggren, H. G., N. J. Stam, J. J. C. Öhlen, P. Neefjes, M. T. Höglund, J. Heemels, T. N. Bastin, A. Schumacher, K. Kärre Townsend, H. L. Ploegh. 1990. Empty MHC class I molecules come out in the cold. Nature 346:476.
March, J., Advanced Organic Chemistry, 4^{th} Edition (1992), Wiley InterScience.
Nikolic-Zugic, J., and Carbone F. R. (1990). The effect of mutations in the MHC Class I peptide binding groove on the cytotoxic T lymphocyte recognition of the Kb restricted ovalbumin determinant. Eur. J. Immunol. 20:2431.
Porgador, A., J. W. Yewdell, Y. Deng, J. R. Bennink, R. N. Germain. 1997. Localization, quantitation, and in situ detection of specific peptide-MHC class I complexes using a monoclonal antibody. Immunity 6:715.
Rotzschke, O. and Falk, K., Naturally-occurring peptide antigens derived from the MHC class-I-restricted processing pathway. Immunol Today. December 1991;12(12):447-55. Review.
Schumacher, N. M., M.-T. Heemels, J. J. Neefjes, W. M. Kast, C. J. Melief, M. H. L. Ploegh. 1990. Direct binding of peptide to empty MHC class I molecules on intact cells and in vitro. Cell 62:563.
Wallace, M. E., Keating, R., Heath, W R., Carbone, F. R. 1999. The cytotoxic T-cell response to herpes simplex virus type 1 infection of C57BL/6 mice is almost entirely directed against a single immunodominant determinant. J Virol. September;73(9):7619-26.
Yuan-Hua Ding, Brian M. Baker, David N. Garboczi. Four A6-TCR/Peptide/HLA-A2 Structures that Generate Very Different T Cell Signals Are Nearly Identical. Immunity, Vol. 11, 45-56, July, 1999.

1. A method of modulating a peptide specific T cell response, said method comprising contacting said T cell with an MHC-peptide complex, which peptide comprises at least one β-amino acid substitution, wherein said β-amino acid substitution induces either agonism or antagonism of said T cell response relative to the T cell response inducible by a non-substituted form of said peptide.

2. A method of modulating a peptide specific T cell response in a subject, said method comprising administering to said subject an effective amount of a peptide, which peptide comprises at least one β-amino acid substitution, for a time and under conditions sufficient to present said peptide to said T cell in the context of an MHC-peptide complex, wherein said β-amino acid substitution induces either agonism or antagonism of said T cell response relative to the T cell response inducible by a non-substituted form of said peptide.

3. The method according to claim 1 wherein said T cell is a CD8⁺ T cell and said MHC is MHC I.

4. The method according to claim 3 wherein said peptide comprises 2-50 amino acid residues.

5. The method according to claim 4 wherein said peptide comprises 2-40 amino acid residues.

6. The method according to claim 5 wherein said peptide comprises 2-30 amino acid residues.

7. The method according to claim 6 wherein said peptide comprises 2-20 amino acid residues.

8. The method according to claim 7 wherein said peptide comprises 2-15 amino acid residues.

9. The method according to any one of claims 1 wherein said response is T cell activation.

10. The method according to claim 9 wherein said activation is agonized.

11. The method according to claim 9 wherein said peptide is a tumour derived peptide.

12. The method according to claim 11 wherein said tumour derived peptide is from NY-ESO, MUC1, MAGE, BAGE, RAGE or CAGE.

13. The method according to claim 9 wherein said peptide is a virus derived peptide.

14. The method according to claim 13 wherein said virus is Epstein Barr Virus, Cytomegalovirus, human immunodeficiency virus or Hepatitis C virus.

15. The method according to claim 9 wherein said peptide is a tolerogenic epitope.

16. The method according to claim 15 wherein said tolerogenic epitope is derived from Myelin Basic Protein (MBP).

17. The method according to claim 1 wherein said amino acid substitutions are substitutions of said peptide's MHC anchor residues.

18. The method according to claim 1 wherein said response is the induction of anergy or tolerance of pathological T cells.

19. The method according to claim 1 wherein said response is the induction of a polyclonal T cell response.

20. A method of agonizing a peptide specific T cell response in a subject, said method comprising co-administering to said subject an effective amount of an antagonistic peptide, which peptide comprises at least one β-amino acid substitution, together with the non-substituted form of said peptide for a time and under conditions sufficient to present said peptides to said T cells in the context.

21. A method of antagonizing a peptide specific T cell response in a subject, said method comprising co-administering to said subject an effective amount of an agonistic peptide, which peptide comprises at least one β-amino acid substitution, together with the non-substituted form of said peptide for a time and under conditions sufficient to present said peptides to said T cells in the context of an MHC-peptide complex.

22. A method for the treatment and/or prophylaxis of a condition characterized by an aberrant, unwanted or otherwise inappropriate peptide specific T cell response in a subject, said method comprising administering to said subject an effective amount of a peptide, which peptide comprises at least one β-amino acid substitution, for a time and under conditions sufficient to present said peptide to said T cell in the context of an MHC-peptide complex, wherein said β-amino acid substitution induces either agonism or antagonism of said T cell response relative to the T cell response inducible by a non-substituted form of said peptide.

23. The method according to claim 22 wherein said T cell is a CD8⁺ T cell and said MHC is MHC I.

24. The method according to claim 23 wherein said peptide comprises 2-50 amino acid residues.

25. The method according to claim 24 wherein said peptide comprises 2-40 amino acid residues.

26. The method according to claim 25 wherein said peptide comprises 2-30 amino acid residues.

27. The method according to claim 26 wherein said peptide comprises 2-20 amino acid residues.

28. The method according to claim 27 wherein said peptide comprises 2-15 amino acid residues.

29. The method according to claim 22 wherein said response is T cell activation.

30. The method according to claim 29 wherein said activation is agonised.

31. The method according to claim 29 wherein said condition is a tumour and said peptide is a tumour derived peptide.

32. The method according to claim 31 wherein said tumour derived peptide is from NY-ESO, MUC1, MAGE, BAGE, RAGE or CAGE.

33. The method according to claim 29 wherein said condition is a viral infection and said peptide is a virus derived peptide.

34. The method according to claim 33 wherein said virus is EBV, CMV, HIV or CIV.

35. The method according to claim 29 wherein said condition is multiple sclerosis and said peptide is a tolerogenic peptide.

36. The method according to claim 35 wherein said tolerogenic epitope is MBP.

37. The method according to claims 22 wherein said response is the induction of anergy or tolerance of pathological T cells.

38. The method according to claims 22 wherein said response is the induction of a polyclonal T cell response.

39. A method for the treatment and/or prophylaxis of a condition characterized by the occurrence of an unwanted peptide specific T cell response in a subject, said method comprising co-administering to said subject an effective amount of an agonistic peptide, which peptide comprises at least one β-amino acid substitution together with a non-substituted form of said peptide for a time and under conditions sufficient to present said peptides to said T cells in the context of an MHC peptide complex.

40. A method for the treatment and/or prophylaxis of a condition characterized by an inadequate peptide specific T cell response in a subject, said method comprising co-administering to said subject an effective amount of an antagonist peptide, which peptide comprises at least one β-amino acid substitution, together with a non-substituted form of said peptide for a time and under conditions sufficient to present said peptides to said T cells in the context of an MHC peptide complex.

41. The method according to claim 39 wherein said condition is an autoimmune condition, a transplant or an allergic condition.

42. The method according to claim 40 wherein said condition is a neoplastic condition or an infection.

43. (canceled)

44. A pharmaceutical composition comprising a β-amino acid substituted peptide together with one or more pharmaceutically acceptable carriers and/or diluents.

45. A method of designing and screening for β-amino acid substituted peptide analogues, which method provides a means of rationally substituting α-amino acids for β-amino acids in a positional scanning approach and the identification of peptides exhibiting desired functional activity and improved bioavailability.
